(19) United States
(12) Patent Application Publication
(10) Pub. No.: US 2026/0297556 A1
(43) Pub. Date: Oct. 1, 2026

(54) Gel Analysis of RNA

(71) Applicant: United Kingdom Research and Innovation, Swindon (GB)

(72) Inventors: Yifei Du, Cambridge (GB); Venkatraman Ramakrishnan, Cambridge (GB)

(21) Appl. No.: 19/490,873

(22) PCT Filed: Jun. 7, 2024
(86) PCT No.: PCT/EP2024/065813

§ 371 Date: Dec. 5, 2025

(30) Foreign Application Priority Data
Jun. 9, 2023 (GB) 2308668.9

(51) Int. Cl. C12N 15/10; C12Q 1/6806

(52) U.S. Cl. CPC C12N 15/101; C12Q 1/6806; C12N 2310/532

(57) ABSTRACT

This invention relates to methods of separating RNA in a sample. Methods may comprise loading an electrophoresis gel with the RNA-containing sample and applying an electric field across the gel to cause the RNA to migrate across the gel, such that the linear and circular RNA present in the sample migrate through the gel at different speeds. The electrophoresis gel may be an urea-agarose gel. Methods of separating circular RNA from linear RNA in a sample are provided, along with methods for analysing RNA present in a sample. Kits for separating linear and circular RNA in a sample are also provided.

This application claims priority from GB2308668.9 filed 9 Jun. 2023, the contents and elements of which are herein incorporated by reference for all purposes.

### FIELD

The invention relates to gel electrophoresis methods for separating circular RNA from its corresponding linear RNA in a sample.

### BACKGROUND

Circular RNAs are covalently closed single-stranded RNA molecules which have no free 5′ or 3′ end. Circular RNAs are being actively explored for development of next-generation mRNA therapeutics, mainly because of their in vitro and in vivo stability.

A number of methods exist to produce synthetic circular RNA. The resulting circular RNA can be identified using a range of techniques including gel electrophoresis, RNase R digestion, oligonucleotide-guided RNase H digestion, single-hit hydrolysis, and high-performance liquid chromatography (HPLC). However, characterization of circular RNA by gel electrophoresis remains challenging. The identification of circular RNA still requires time-consuming processes, particularly for circular RNA of up to 6,000 nucleotides.

Native agarose gel has been found to give good separation based on RNA length but is not able to resolve circular RNA from its linear counterpart (Zhang Y et al (2016)) at all lengths. Attempts to resolve RNA in samples in native agarose gel has found that circular RNA can co-migrate with their linear counterparts.

Denaturing polyacrylamide gels (PAGE) have been found to distinguish circular RNAs from their linear counterparts as circular RNAs migrate much slower than their linear counterparts. But this system is not suitable for RNAs longer than about 500 nucleotides. Wesselhoeft R et al (2018) have found that the E-gel system (a prepackaged native agarose gel system, Thermo Fisher) is able to separate circular RNAs and their linear counterparts, but unfortunately, these gels show batch to batch variation. Abe B T et al (2022) also consider the use of native agarose gel systems on the migration patterns of circular RNA.

Formaldehyde is used in some system as the denaturing reagent to provide denaturing gels. However, formaldehyde is toxic and formaldehyde-agarose gels can be difficult to handle.

Urea is also used as a denaturing agent, however whilst it is considered to work well as the denaturing reagent in PAGE, it is not considered suitable for use in agarose gels, because urea is a weak denaturant and requires a high concentration (above 4M) to work (Masek, T et al (2005), Sumitomo, K et al (2009)). A high concentration of urea is known to reduce the physical strength of agarose. Urea-agarose gels with high concentrations of urea, are considered fragile and difficult to handle, and therefore are not often used.

Therefore, there is a need for alternative systems for circular RNA analysis, in particular methods that are suitable of distinguishing circular RNA from linear RNA over a range of lengths.

### SUMMARY

Accordingly, a first aspect of the invention provides a method of separating RNA present in a sample, the method comprising:
(a) loading an electrophoresis gel with the RNA-containing sample, wherein the electrophoresis gel is an urea-agarose gel; and
(b) applying an electric field across the gel to cause the RNA to migrate across the gel, wherein linear and circular RNA present in the sample migrate through the gel at different speeds.

In a preferred embodiment of the invention the urea-agarose gel comprises urea at a concentration of about 1M to about 8M. In a further embodiment of the invention the urea-agarose gel is a 0.5% to 6% agarose gel.

A second aspect of the invention provides a method for separating circular RNA from linear RNA in a sample, the method comprising electrophoretically separating RNA in sample in a gel, wherein the gel is a denaturing urea-agarose gel and positioned in a vertical arrangement.

A third aspect of the invention provides a method for analysing RNA present in a sample, the method comprising analysing the sample by gel electrophoresis, wherein the wherein the gel is a denaturing urea-agarose gel and wherein the method comprises using a vertical gel electrophoresis system.

In a fourth aspect the invention provides a kit for separating linear and circular RNA in a sample, the kit comprising:
(a) components for carrying out gel electrophoresis of a RNA containing sample, wherein the components include agarose and urea for the preparation of a urea-agarose gel as defined herein;
(b) a loading buffer for mixing with the RNA sample, wherein the buffer comprises urea as a denaturing agent; and
(c) a standard molecular weight control sample.

Further aspects and embodiments of the invention are described below.

### BRIEF DESCRIPTION OF FIGURES

FIG. 1: Analysis of Circular CVB3-EGFP and its linear counterpart in 0.8% native agarose gel. (A) In vitro transcription (IVT) of the CVB3-EGFP was performed in the presence of 10, 14, 18, 24 mM Mg²⁺. ~500 ng of each IVT product was loaded to a 0.8% native agarose gel for a 70 min running at constantly 25W. Full length precursor of CVB3-EGFP is 1963 nucleotides and the circular/nicked products are 1638 nucleotides. Two bands can be seen at the position of the circular/nicked CVB3-EGFP. (B) Full length CVB3-EGFP and circularized CVB3-EGFP were treated with RNase R and loaded onto a 0.8% native agarose gel for a 70 min running at 25W. (C) Gel purified circular CVB3-EGFP was linearized with RNase H in the presence of a 34 nucleotide DNA primer. RNase H digests RNA that hybrids with DNA. Samples were resolved in a 0.8% native agarose gel for an 83 min running at 25W. (D) RT-PCRs were performed on full length CVB3-EGFP and circularized CVB3-EGFP. The presence of a 922 bp product from circularized CVB3-EGFP confirms the circularization.

FIG. 2: Analysis of full-length precursor and circularized RNA in native agarose gels. Full length precursors and circularized RNAs were loaded to (A) 0.8%, (B) 1.5% and (C) 3.0% native agarose gels for running at 25W at indicated time (A) 83 min (B) 85 min and (C) 85 min. (D) Circularized RNA were digested with RNase R and resolved in a 3% native agarose gel for indicated running time (25W for 120 min).

FIG. 3: Analysis of circular RNAs and their linear counterparts in formaldehyde agarose gels. Full length precursors and circularized RNAs were loaded to (A) 0.8% and (B) 1.5% denaturing formaldehyde agarose gels for running at indicated time (8W for 90 min).

FIG. 4: Analysis of circular RNAs and their linear counterparts in horizontal urea agarose gels. Full length precursors and circularized RNAs were loaded to a 1.5% agarose gels with 6M urea. Gels were run in a horizontal electrophoresis system with indicated settings (20W for 40 min).

FIG. 5: Analysis of circular RNAs and their linear counterparts in vertical urea agarose gels. Full length precursors and circularized RNAs were loaded to either 1.5% agarose gels with (A) 6M urea, (B) 4M urea or (C) 2M urea, or 0.8% agarose gel with (D) 6M or (E) 2M urea. (F) Circularized 3*Flag and its RNase R digested product were load to a 6M urea 4% agarose gel. Full length precursor and circular 3*Flag are 525 and 212 nucleotides, respectively. Gels were run in a vertical electrophoresis system with indicated settings (20W for (A) 29 mins, (B) 26 min, (C) 18 min, (D) 23 min, (E) 11 min and (F) 25 min).

FIG. 6: Analysis of circular RNAs and their linear counterparts in urea agarose gels. 6M-1.5% urea agarose gels were tested in various running settings. (A)-(B) Running power was fixed at 20W and running time of 24 min and 28 min were compared. (C)-(D) Running time was fixed at 30 min and running power of 20W and 15W were compared. Loading buffer used in (A) and (B) is the denaturing urea loading buffer while in (C) and (D) is denaturing formamide loading buffer.

### DETAILED DESCRIPTION

The inventors have found that when urea is used as the denaturing reagent in an agarose electrophoresis gel system, circular RNAs migrate slower than their linear counterparts, regardless of RNA length and agarose concentration.

Accordingly, the invention provides a method for separating circular RNA from linear RNA in a sample, the method comprising electrophoretically separating RNA in sample in a gel, wherein the gel is a denaturing urea-agarose gel.

In one aspect the invention provides a method for separating RNA present in a sample by gel electrophoresis. The method for separating RNA present in a sample comprises:
(a) loading an electrophoresis gel with the RNA-containing sample, wherein the electrophoresis gel is an urea-agarose gel; and
(b) applying an electric field across the gel to cause the RNA to migrate across the gel. The linear and circular RNA present in the sample migrate through the gel at different speeds.

The method separates circular RNA from its counterpart linear RNA in a sample. The wells of the prepared denaturing urea-agarose gels are loaded with the samples to be tested. The gel is then run at a power and for a time period that will result in separation of the circular RNA from its counterpart linear RNA. The RNA can then be visualised by appropriate means, for example by staining and imaging.

The inventors have found that denaturing urea/agarose gels are effective at separating circular RNA from its linear counterpart in a sample. Despite the previous known issues using urea as a denaturing agent and with agarose gels, when urea is used as a denaturing agent in the preparation of the agarose gel, the gels are capable of separating long circular RNA from its linear counterparts in a simple and quick method. The methods of the invention do not require long run times to separate the circular RNA from its linear counterpart.

In some embodiments of the invention the urea can be present in the agarose gel at a concentration of up to about 1M to 8M, e.g. at a concentration of about 1M, about 2M, about 3M, about 4M, about 5M, about 6M, about 7M or about 8M in the gel. In some embodiments the urea is present at a concentration in the range of about 2M to about 8M, about 2M to 6M, about 2M to 4M or about 4M to 6M in the gel. In one embodiment the urea is present in the agarose gel at a concentration of about 4M-6M. In one embodiment the urea is present in the agarose gel at a concentration of about 4M. In one embodiment the urea is present in the agarose gel at a concentration of about 6M.

The concentration of urea will depend on the size of RNA intended to be separated. Lower concentrations of urea can be used if the intention is to separate longer RNA. For example, in some embodiments of the invention wherein the intention is to separate RNA having a length above 1000 nucleotides urea can be present in a concentration of about 2M. However, urea can still be present in higher concentrations if the intention is to separate RNA's greater than 1000 nucleotides. For example, wherein the intention is to separate RNA greater than 1000 nucleotides, gels wherein the urea is present in concentrations of about 4M or 6M can still be used. For example, if the intention is to also separate short RNA's e.g. less than 500 nucleotides, in the same gel then urea can be present in higher concentrations. Wherein the intention is to separate RNA having a length less than 500 nucleotides, urea can be present in concentrations of about 6M.

In some embodiments, wherein the method is to separate RNA in a sample, or multiple samples on one gel, wherein a sample or samples comprise RNA in a range of lengths from less than 500 (e.g. from about 200 nucleotides) to about 6000 nucleotides, then preferably a concentration of 6M of urea can be used.

References herein to “gels”, “gel” or similar may alternatively be expressed as “electrophoresis gels” or similar. Methods of making electrophoresis gels are known in the art. In particular, agarose electrophoresis gels for separating biomolecules such as nucleic acids are known in the art. Sources of agarose and techniques for preparing and running such gels are disclosed in references textbooks such as Green and Sambrook (Molecular cloning: a laboratory manual, 4th edition, New York: Cold Spring Harbor Laboratory, 2012) and Ausubel et al eds. (Current Protocols in Molecular Biology, Wiley, 2003), and reference cited herein. The contents of which are incorporated herein by reference. Such techniques may be adapted for use in accordance with the present invention. For example, to produce gels with the required agarose and urea concentrations.

In some embodiments of the invention the agarose is present in the gel in a concentration of 0.5% to 6% agarose. In some embodiments the agarose is present in a concentration of about 0.5% to about 5% agarose. In some embodiments the agarose is present in a concentration of about 0.8% to about 2% agarose. In some embodiments the agarose is present in a concentration of about 0.8%, 1%, 1.5%, 2%, 2.5%, 3%, 3.5%, 4%, 5% or 6% agarose. In one embodiment the agarose is present in a concentration of about 1.5%. i.e. the gel is a 1.5% agarose gel. The concentration of agarose in gels is measured as weight in agarose over volume of buffer used (w/v %).

The concentration of agarose can depend on the size of RNA intended to be separated. Lower concentrations of agarose can be used if the intention is to separate longer RNA. For example, in wherein the intention is to separate RNA above 1000 nucleotides agarose is present in a concentration of about 0.8% to 2% agarose, preferably 1.5%. If the intention is to also separate short RNA's e.g. less than 500 nucleotides, then the agarose can be present in higher concentrations of agarose. For example, wherein the intention is to separate RNA less than 500 nucleotides, agarose can be present in a concentration of about 1.5% to about 4%.

In some embodiments, wherein the method is to separate RNA in a sample, or multiple samples on one gel, wherein the sample or samples comprise RNA in a range of lengths from less than 500 to about 6000 nucleotides, then preferably agarose is present at a concentration of about 1.5%.

In some embodiments the urea-agarose gels used in the methods of the invention may comprise urea at a concentration in the range of 1M to 8M and agarose at a concentration of about 0.5% to 6%, urea in the range of 2M to 8M and agarose in the range of 0.8% to 5%, urea in the range of 2M to 6M and agarose in the range of 0.8% to 5%, urea in the range of 2M to 4M and agarose in the range of 0.8% to 2%, or urea in the range of 4M to 6M and agarose in the range of 0.8% to 2%, urea in the range of 2M to 4M and agarose at about 1.5%, or urea in the range of 4M to 6M and agarose at about 1.5%.

Accordingly, in some embodiments of the invention a 2M urea/0.8% agarose gel is used in the methods. In some embodiments a 2M urea/1.5% agarose gel is used in the methods. In some embodiments a 4M urea/1.5% agarose gel is used in the methods. In some embodiments a 6M urea/1.5% agarose gel is used in the method. In some embodiments a 6M urea/4% agarose gel is used in the methods.

The separation of the RNA in the sample can depend on the power and time period that the gel is run. Standard systems known in the art can be used to provide generating the electrical field across the gel. In some embodiments of the invention electrophoresis may be carried out a constant power in the range of 5W-75W. However higher or lower power can be used. In some embodiments the electrophoresis may be carried out a constant power in the range of 15W-50W, for example at a constant power of about 5W, 10W, 15W, 20W, 25W, 30W, 35W, 40W, 45W or 50W. In some embodiments the electrophoresis may be carried out at a constant power in the range of 15-40W, in the range of 15W-30W or in the range of 15-25W. Preferably the electrophoresis is carried out at a constant power in the range of about 15-25W. In one embodiment the electrophoresis is carried out at a constant of about 15W. In another embodiment electrophoresis is carried out at a constant power of about 20W.

In some embodiments of the invention the electrophoresis can be run for a time period of about 10-90 minutes. However longer or short running times can be used, In some embodiments of the invention the electrophoresis can be run for a time period of about 10-60 minutes, for example for a time period of about 10 minutes, about 15 minutes, about 20 minutes, about 25 minutes, about 30 minutes, about 35 minutes, about 40 minutes, about 45 minutes, about 50 minutes, about 55 minutes, or about 60 minutes. In some embodiments the electrophoresis can be run for a time period of about 10-40 minutes, about 10-30 minutes, about 15-30 minutes, about 20-30 minutes or about 10-20 minutes. In some embodiments electrophoresis is run for time period of less than 60 minutes. Preferably the electrophoresis is run for time period of 10-30 minutes.

An advantage of the present invention is that shorter running times can used, whilst still providing sufficient separation between circular and counterpart linear RNA in a sample.

In some embodiments electrophoresis is carried out at room temperature (e.g. in the range of 20-25° C.). In some embodiments electrophoresis is carried out at a constant power in the range of about 15-25W for 10-30 minutes at room temperature.

The running time and power can depend on the size of RNA intended to be separated. Higher running power and/or longer running time may be used if the intention is to separate shorter RNA. For example, in some embodiments of the invention wherein the intention is to separate RNA less than 500 nucleotides the running power can be about 25W for more than about 30 minutes.

If the intention is to separate long RNA's e.g. greater than 500 nucleotides, preferably greater than 1000 nucleotides, shorter running time and/or lower power can be used. For example, in some embodiments of the invention wherein the intention is to separate RNA above 1000 nucleotides the running power can be a constant power in the range of 15-25W for about 10-30 minutes. In some embodiments of the invention wherein the intention is to separate RNA above 1000 nucleotides the running power can be about 20W for about 10-30 minutes.

The urea-agarose gels are suitable for separating long circular RNA from its linear counterparts in sample. The term “circRNA” or “circular RNA” or similar refer to a polyribonucleotide that forms a circular structure through covalent or non-covalent bonds. References to a linear counterpart sequence or similar means the linear RNA sequence has the same or similar nucleotide sequence, in particular for the sequence encoding the gene of interest but is not circularized and has two free ends.

In some embodiments the linear RNA counterpart sequences, is the linear precursor sequence before circularization. By “long RNA” or similar it is meant RNA having a length greater than 500 nucleotides.

In some embodiments of the invention the methods are capable of separating circular RNA from its linear counterpart, when the RNA has a length up to about 7000 nucleotides. In some embodiments the methods can separate RNA having a length of up to about 1000 nucleotides, up to about 2000 nucleotides, up to about 3000 nucleotides, up to about 4000 nucleotides, up to about 5000 nucleotides, up to about 6000 nucleotides, or up to about 6500 nucleotides. In some embodiments the methods can separate RNA having a length of up to about 6000 nucleotides.

Although the methods of the inventions are particularly suitable for separating long circular RNA from its linear counterparts, in some embodiments of the method is also for separating short circular RNA from its linear counterpart. By “short” RNA it is meant RNA having a length less than 500 nucleotides. Preferably the urea-agarose gels can be used to separate RNA having a length longer than about 200 nucleotides, longer than about 300 nucleotides, longer than about 400 nucleotides or longer than about 500 nucleotides.

Preferably the methods of the invention are for separating RNA having a length in the range of about 200 nucleotides to about 7000 nucleotides, in the range of about 200 nucleotides to about 6500 nucleotides, in the range of about 200 nucleotides to about 6000 nucleotides, in the range of about 500 nucleotides to about 6000 nucleotides, in the range of about 500 nucleotides to about 6500 nucleotides, in the range of about 1000 nucleotides to about 6000 nucleotides, in the range of about 1000 nucleotides to about 6500 nucleotides, in the range of about 1500 nucleotides to about 6000 nucleotides and in the range of about 1500 nucleotides to about 6500 nucleotides. In some embodiments the methods are for separating RNA having a length in the range of about 200 nucleotides to about 6000 nucleotides.

In some embodiments the methods are for separating RNA having a length of up to about 6000 nucleotides, wherein the urea is present in the gel at a concentration of 2M-6M and the agarose is present in a concentration of about 0.8%-4%. Preferably the gel is an 1.5% agarose gel.

In some embodiments of the invention, the methods are for separating RNA having a length in the range of about 500 nucleotides to about 6000 nucleotides, wherein the urea is present in the gel at a concentration of 2M-6M and the agarose is present in a concentration of about 1.5%. In some embodiments of the invention, the methods are for separating RNA having a length in the range of about 500 nucleotides to about 6000 nucleotides, and a 2M-4M urea/1.5% agarose gel is used. In some embodiments of the invention, the methods are for separating RNA having a length in the range of about 500 nucleotides to about 6000 nucleotides, and a 4M-6M urea/1.5% agarose gel is used.

In some embodiments of the invention, the methods are for separating RNA having a length in the range of about 500 nucleotides to about 6000 nucleotides, wherein the urea is present in the gel at a concentration of 2M-6M, the agarose is present in a concentration of about 1.5%, the running power is at a constant power in the range of 15-25W and the running time is about 10 to 30 minutes. In some embodiments of the invention, the methods are for separating RNA having a length in the range of about 500 nucleotides to about 6000 nucleotides, a 2M-4M urea/1.5% agarose gel is used, the running power is at a constant power in the range of 15-25W and the running time is about 10 to 30 minutes. In some embodiments of the invention, the methods are for separating RNA having a length in the range of about 500 nucleotides to about 6000 nucleotides, a 4M-6M urea/1.5% agarose gel is used, the running power is at a constant power in the range of 15-25W and the running time is about 10 to 30 minutes.

In some embodiments of the invention, the methods are for separating RNA having a length in the range of about 1000 nucleotides to about 6000 nucleotides, a 4M-6M urea/0.8%-1.5% agarose gel is used, the running power is at a constant power in the range of 15-25W and the running time is about 10 to 30 minutes. In some embodiments of the invention, the methods are for separating RNA having a length in the range of about 1000 nucleotides to about 6000 nucleotides, a 4M urea/1.5% agarose gel is used, the running power about 20W and the running time is about 10 to 30 minutes.

Electrophoresis gels can be run in a vertical or horizontal arrangement. Typically, agarose gels are operated in a horizontal arrangement. Horizontal gel electrophoresis systems are known in the art. Systems for running the gels in a horizontal arrangement include but are not limited to the E-Gel™ Power Snap Plus Electrophoresis Systems (Invitrogen). However, in some embodiments of the invention the urea-agarose gel is in a vertical position during electrophoresis. The method can comprise using a vertical electrophoresis gel system. Positioning the gel in a vertical arrangement can improve the resolution of the bands, and enhance the separation between the circular and linear RNA in a sample, as compared to running the gel in a horizontal arrangement. An improved resolution allows sharper or narrower bands of molecules, distanced or spaced apart from each other as compared to bands achieved via other means of separation.

Vertical gel electrophoresis system are known in the art, such systems include Bio-Rad Mini-PROTEAN Tetra Vertical Electrophoresis Cell, Owl™ Dual-Gel Vertical Electrophoresis System (Thermo Scientific), XCell SureLock™ Mini-Cell (Invitrogen). In vertical gel electrophoresis systems, the gel is supported in a frame in a substantially vertical position during electrophoresis between a bottom plate and a cover plate. An electrical field is created vertically across the gel. Typically, vertical gel electrophoresis system are for use with PAGE electrophoresis. These systems can be adapted to improve their use with agarose gels. For example, in some embodiments a stopper may be positioned at the bottom edge of the bottom plate. The stopper can help stop the gel slipping during electrophoresis.

In some embodiments, the method further comprises:
running in a first lane of the electrophoresis gel a control sample comprising molecular weight size markers;
running in a second lane of the electrophoresis gel the RNA sample; and
visualising the bands.

The method can further comprise comparing the mobility and banding patterns of the first and second lane to determine if a circular and/or linear RNA is present.

The bands in the gel can be visualised by appropriate means known in the art. The means of visualising the bands will depend on the dye used in the methods. The bands can be visualised using UV imaging techniques. Visualising the bands can comprise staining the gel and visualising the bands using UV imaging techniques. Methods for gel staining and gel imagining are known in the art.

In one aspect of the invention is a method for separating circular RNA from linear RNA in a sample, the method comprising electrophoretically separating RNA in sample in a gel, wherein the gel is a denaturing urea-agarose gel and positioned in a vertical arrangement. The gels and operating conditions for use in the electrophoresis are as described above. For example, in some embodiments the urea-agarose gel, comprises urea in a concentration of 1M to 8M and agarose at a concentration of about 0.5% to 6%. Preferably the urea-agarose gel, comprises urea in a concentration of 4M to 6M and agarose at a concentration of about 1.5%. Preferably the method is for separating RNA having a length up to about 6000 nucleotides.

In a further aspect of the invention there is a method for analysing RNA present in a sample. The method comprises analysing the sample by gel electrophoresis, wherein the gel is a denaturing urea-agarose gel and wherein the method comprises using a vertical gel electrophoresis system. The gels and operating conditions for use in the electrophoresis are as described in the above embodiments. For example, in some embodiments the urea-agarose gel, comprises urea in a concentration of 1M to 8M and agarose at a concentration of about 0.5% to 6%. Preferably the urea-agarose gel, comprises urea in a concentration of 4M to 6M and agarose at a concentration of about 1.5%. Preferably the method is for analysing RNA having a length up to about 6000 nucleotides.

In some embodiments the method is for length analysis and/or form analysis of the RNA. By form analysis it is meant to determine whether circular and/or linear RNA is present.

The methods of invention are particularly useful for complicated RNA containing samples, for example samples comprising different types of RNA species with multiple lengths. For example, a sample may comprise circular RNA, and linear RNA. The sample may also comprise RNA of multiple lengths. The methods of the inventions are able to resolve the circular RNA from the linear RNA that may be present in the sample. Therefore, in some embodiments of the invention, the sample comprises linear and circular RNA. The sample may also comprise RNA of different lengths.

By multiple types or species of RNA, it is meant two or more different forms of RNA, such as circular RNA and linear RNA. The presence of circular RNA in the sample can be identified, as it will migrate through the gel at a different speed than other RNA species in the sample. As such the methods of the invention can be used to detect whether circular RNA is present in a sample.

In a further aspect of the invention there is a method for determining the presences of circular RNA in a sample. The method comprises analysing the sample by gel electrophoresis, wherein the gel is a denaturing urea-agarose gel. The gel may be positioned in a vertical arrangement during electrophoresis. The gels and operating conditions for use in the electrophoresis are as described in the above embodiments. For example, in some embodiments the urea-agarose gel, comprises urea in a concentration of 1M to 8M and agarose at a concentration of about 0.5% to 6%. Preferably the urea-agarose gel, comprises urea in a concentration of 4M to 6M and agarose at a concentration of about 1.5%. Preferably the method is for determining whether circular RNA having a length up to about 6000 nucleotides is present in sample.

The method can be used to detect whether circular RNA was produced, following in vitro or in vivo production of circular RNA using standard methods in the art. A sample of the resultant product from the production method can be analysed using the methods of the invention to determine whether the circular RNA was produced, by detecting whether circular RNA is present in the sample. The circular RNA will migrate at a different speed than the corresponding linear RNA, enabling detection of the circular RNA. In a urea-agarose gel the circular RNA will migrate more slowly than its corresponding linear counterpart.

Therefore, in one embedment the invention comprises a method for determining the presence of circular RNA in a sample. The method comprises:
(a) loading an electrophoresis gel with the sample, wherein the electrophoresis gel is a urea-agarose gel; and
(b) applying an electric field across the gel to cause the RNA present in the sample to migrate across the gel;
(c) analysing the mobility and band pattern of the gel to determine the presence of circular RNA in the sample.

The gels and operating conditions for use in the method are as described in the above embodiments.

The urea-agarose gels for use in the methods of the invention can be prepared by known techniques as discussed above.

Agarose gels can be formed by suspending dry agarose in an aqueous, usually buffered, media, and heating the mixture to melt the agarose in the usual manner. The agarose containing medium is mixed with urea at the appropriate concentration. The urea-agarose media can be mixed with further buffer and aqueous media and then poured into a cassette and cooled to allow the gel to set. A plurality of wells can be introduced into a gel, by using a “comb” having a row of protruding teeth positioned so that the teeth project into the gel layer while it sets. Cooling can occur a room temperature (e.g. in the range of 20-25° C.) or at a reduced temperature for example at a temperature in the range of 4-6° C.

In some embodiments the gel is prepared by mixing an aqueous media with agarose at the appropriate concentration to provide a mixture with agarose in the desired concentration. In some embodiments the aqueous media is DEPC treated water. Other suitable aqueous medias can be used.

Therefore, in some aspects of the invention the denaturing urea/agarose gel is prepared by: (a) mixing agarose with DEPC treated water and heating the mixture to dissolve the agarose and form an aqueous agarose mixture; the appropriate amount of agarose is mixed to provide an agarose gel with the desired agarose concentration;
(b) mixing urea with the aqueous agarose mixture of (a), to provide a urea-agarose mixture; the appropriate amount of urea is mixed to provide an urea/agarose gel with the desired urea concentration.
(c) the urea-agarose mixture from (b) is mixed with an electrophoresis buffer, and further DEPC treated water;
(d) pouring the resultant mixture from (c) into gel plates; and
(e) allowing the gel to set over a period of time,
wherein the resultant gel comprises urea in a concentration in the range of 1M to 8M and agarose in the range of 0.5% to 6%.

In one embodiment, in step (c) a dye capable of binding RNA is also mixed with the urea-agarose mixture.

In some embodiments the RNA binding dye is not mixed with the urea-agarose mixture before casting the gels. In such embodiments the gels are stained after electrophoresis with a mixture of the electrophoresis buffer and RNA-binding dye.

The buffer used during the staining can be a urea-free buffer. By urea-free it is meant that the buffer does not contain urea. Using a urea-free buffer during staining, can help the strength of the gel.

Buffers for use in staining gels that do not comprise urea are known in the art, and include but are not limited to, TBE (Tris-Borate-EDTA buffer) and TAE (Tris-acetate-EDTA buffer).

In some embodiments, the gel plates are preheated before the gel mixture is poured into the gel plates. Preheating the plates helps avoid gelation during pouring of the gel mixture. Preferably the plates are preheated wherein the gel comprises urea in a concentration of about 6M and agarose is in a concentration of about 4%.

The test sample can be any sample containing RNA. Methods for preparing the test sample are known in the art.

The test sample can be denatured before loaded into the gel. The RNA containing sample may be prepared by mixing the sample with loading buffer, preferably a denaturing loading buffer. Examples of denaturing loading buffers are known in the art for example, denaturing formamide loading buffers and denaturing urea loading buffers. Preferably the loading buffer comprises urea as a denaturing agent. Examples of urea loading buffers are known in the art. In some embodiments of the invention the RNA sample may be mixed with an equal volume of urea loading buffer. The mixture can then be denatured at 95° C. for about 2 minutes, before loading the samples into the wells of the gels.

Suitable electrophoresis buffers, dyes, loading buffers and other aqueous mediums known in the art can be used to produce the urea-agarose gel. For example, electrophoresis buffers include but are not limited to TAE and TBE, and RNA binding dyes include but are not limited to SYBR Green and ethidum bromide.

In some embodiments the gel is not formed from acrylamide, e.g. the gel is not a composite polyacrylamide-agarose gel. In some embodiments the gel does not comprise formaldehyde as a denaturing agent. In some embodiments formaldehyde and/or formamide may also not be present in the electrophoresis buffer or the loading buffer.

The invention also provides a kit for separating linear and circular RNA in a sample, the kit comprising;
(a) components for carrying out gel electrophoresis of a RNA containing sample, wherein the components include agarose and urea for the preparation of a urea-agarose gel as described above;
(b) a buffer for mixing with the RNA sample, wherein the buffer comprises urea as a denaturing agent; and
(c) a standard molecular weight control sample.

The standard molecule weight control sample comprises suitable molecular weight size markers. The kits may further comprise a urea-free staining buffer. The kit may further comprise an apparatus for vertical gel electrophoresis.

The terms “comprising” or “comprises” may be substituted with the terms “consisting of”, “consists of”, “consisting essentially of” or “consists essentially of” and vice versa, wherever they occur herein.

Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

The contents of all publications cited herein are incorporated herein by reference in their entirety into this application to more fully describe the state of the art to which this invention pertains.

The present invention will be further understood by reference to the following examples.

### EXAMPLES

In the Examples provided below it is shown that the use of urea as the denaturing agent improved the separation of circular RNA from its linear counterparts in agarose gel electrophoresis.

### Materials and Methods

### Plasmids

The PIE Ana 3.0 circularization construct that lacks spacers, internal homology arms and gene of interest (GOI) was placed between a T7 promoter and Not I and EcoR V cleavage sites and synthesised from the IDT (Integrated DNA Technologies, pUCIDT-PIE-Ana3.0-empty) (Wesselhoeft, R. A. et al 2018). GOIs and corresponding backbone vectors were PCR amplified separately and assembled by the Gibson assembly master mix (NEB). All plasmids were Sanger sequenced and amplified by homemade TOP10 competent cells. For CVB3-EGFP (pUCIDT-PIE-Ana3.0-CVB3-EGFP), a gene block contains exons, spacers, internal homology arms and CVB3-EGFP was synthesised from the IDT and amplified by primers CVB3-EGFP-gF and CVB3-EGFP-gR. Vector for CVB3-EGFP was amplified from the pUCIDT-PIE-Ana3.0-empty by primers empty-vF and empty-vR. Then the pUCIDT-PIE-Ana3.0-CVB3-EGFP was used as the backbone vector for Firefly luciferase, Spike protein, Cas9 and T2A-EGFP. The plasmid for circular 3*Flag was synthesis by IDT. All plasmids were scaled up by the plasmid plus maxi kit (QIAGEN) and linearized by EcoR V (NEB) or Not I (NEB) for following in vitro transcription (IVT). Primers for vectors and these genes are listed in Table 1 and 2.

TABLE 1

| | SEQ ID NO |
|---|---|
| pUCIDT-PIE-Ana3.0-empty | SEQ ID NO: 1 |
| pUCIDT-PIE-Aan3.0-CVB3-EGFP | SEQ ID NO: 2 |
| pUCIDT-PIE-Aan3.0-CVB3-Fluci | SEQ ID NO: 3 |
| pUCIDT-PIE-Aan3.0-CVB3-Spike-EGFP | SEQ ID NO: 4 |
| pUCIDT-PIE-Aan3.0-CVB3-Cas9-EGFP | SEQ ID NO: 5 |
| pUCIDT-PIE-Aan3.0-T2A-EGFP | SEQ ID NO: 6 |
| pUCIDT-PIE-Aan3.0-3*Flag | SEQ ID NO: 7 |

TABLE 2

| Primers for | | Sequence | SEQ ID NO: |
|---|---|---|---|
| CVB3- | empty-vF | AGACGCTACGGACTTAAATAATTGAGC | 8 |
| EGFP | empty-vR | GTTTAAGGTCAACGGATTTTCATTCTC | 9 |
| | CVB3-EGFP-gF | AAAATCCGTTGACCTTAAACGGTCG | 10 |
| | (F1) | | |
| | CVB3-EGFP-gR | AAGTCCGTAGCGTCTCGCC | 11 |
| CVB3- | CVB3-Fluci-gF | GTTGAATACAGCAAAATGGAAGATGCCA | 12 |
| Firefly | | AAAACATTAAGAAGG | |
| Luciferase | CVB3-Fluci-gR | TTTGTTTTTTTTACTTATCATCATCATCCTT | 13 |
| | | GTAATCACTGCTCACGGCGATCTTGCCGC | |
| | CVB3-Fluci-vF | AGTAAAAAAAACAAAAAACAAAACGGCTAT | 14 |
| | (F2) | TATGCGTTACCGGCGAGACGCTACGGACTT | |
| | CVB3- | TTTGCTGTATTCAACTTAACAATGAATTGTA | 15 |
| | Fluci/Spike/Cas9- | ATG | |
| | vR (R) | | |
| CVB3- | CVB3-Spike- | GTTGAATACAGCAAAATGGGGATTCTGCCTA | 16 |
| Spike/Cas9- | EGFP-gF | GCCC | |
| EGFP | CVB3-Spike- | ACTGCTGCCGCTGCCCAAGAAGGTGCTCAG | 17 |
| | EGFP-gR | CAGCAC | |
| | CVB3-Cas9- | GTTGAATACAGCAAAATGGATAAGAAATACT | 18 |
| | EGFP-gF | CAATAGGCTTAGATATC | |
| | CVB3-Cas9- | ACTGCTGCCGCTGCCGTCACCTCCTAGCTG | 19 |
| | EGFP-gR | ACTCAAATC | |
| | Spike/Cas9-vF | GGCAGCGGCAGCAGTATGGTGAGCAAGGG | 20 |
| | | CGAGG | |
| T2A-EGFP | T2A-EGFP-gF | CTGCTAAAGCAAGCAGGTGATGTTGAAGAA | 21 |
| | | AACCCCGGGCCTGGTACTCCCATCATGGTG | |
| | | AGCAAGGGCGAG | |
| | T2A-EGFP-gR | ATTATTTAAGTCCGTCTTGTACAGCTCGTCC | 22 |
| | | ATGC | |
| | T2A-EGFP-vF | ACGGACTTAAATAATTGAGCC | 23 |
| | T2A-EGFP-vR | TGCTTGCTTTAGCAGAGAGAAGTTTGTGGC | 24 |
| | | GCCGCTGCCAACGGATTTTCATTCTCTTGC | |

The GOIs used in the following experiments have the following precursor/circular lengths:

TABLE 3

| | Precursor | Circular |
|---|---|---|
| GOI | length | length |
| CVB3-EGFP | 1963 nucleotides | 1638 nucleotides |
| CVB3-Firefly luciferase | 2925 nucleotides | 2600 nucleotides |
| (Fluci) | | |
| CVB3-Spike-EGFP | 5793 nucleotides | 5468 nucleotides |
| CVB3-Cas9-EGFP | 6081 nucleotides | 5756 nucleotides |
| (CVB3-Cas9) | | |
| T2A-EGFP | 1138 nucleotides | 813 nucleotides |

Circular RNA Synthesis IVT were performed at 50 ng/μl of DNA template, 14 μg/μl of homemade T7 polymerase, 0.04 U/μl of RNase inhibitor (Promega), 6 mM of each NTPs and 1X IVT buffer. For IVT where co-transcriptional splicing is allowed, 1X IVT buffer contains 80 mM Tris-HCl (pH 7.4), 2 mM spermidine, 40 mM DTT and 24 mM MgCl₂. To test the dependence of co-transcriptional splicing on MgCl₂, concentration of MgCl₂ in 1X IVT buffer was 10, 14, 18, or 24 mM. When co-transcriptional splicing is to be suppressed, the concentration of MgCl₂ in 1X IVT buffer is 14 mM. IVT reactions were incubated at 37° C. for 3-5 h and then digested by RNase-free DNase I for 20 min. Afterwards, 100 mM EDTA was added to a concentration of 25 mM to clear any precipitation. Then equal volume of 7.5 M lithium chloride is added to precipitate RNAs for 30 min to overnight at −20° C. Then precipitations were spun down at 13000 rpm/min for at least 20 min. RNA pellets were washed by 75% alcohol, air dry and dissolved in DEPC treated H₂O.

For circularization, RNA precursors were firstly diluted to ~700 ng/μl. Then 90 μl of each RNA was denatured at 95° C. for 2 min and annealed on ice for 3 min. Then annealed RNAs were supplied with 10 ul of 10X circularization buffer (500 mM Tris-HCl, pH=7.4, 100 mM MgCl₂, 10 mM DTT, 20 mM GTP) and heated at 55° C. for 20 min. Circularization was stopped by adding 20 μl of 100 mM EDTA.

### RNase R and RNase H Digestion

To do RNase R digestion, circularized CVB3-EGFP were firstly column cleaned (ZYMO RESEARCH). Then 5 ug of full-length precursor and 5 μg of circularized RNA were digested with 10 U of RNase R (antibodies-online) at 37° C. for 15 min. Digested RNAs were loaded to native agarose gel as described below.

To do RNase H digestion, 100 μg of CVB3-EGFP full length precursor RNA was circularized and separated in a 0.8% native agarose gel for 80 min, then circular RNA band was cut and extracted by an RNA gel recovery kit (ZYMO RESEARCH). Circular RNA was eluted in DEPC H₂O. Then 5 ug purified circular CVB3-EGFP was heated in the presence of 5 times molar amount of the CVB3-Fluci/Spike/Cas9-vR primer at 65° C. for 5 min. After 3 min of cooling down on ice, RNase H (NEB) and buffer was added for digestion at 37° C. for 20 min. Digested RNA was loaded to a native agarose gel as described below.

### RT-PCR

Reverse transcriptase and DNA polymerase used here are the SuperScrip IV Reverse Transcriptase (Thermo Fisher) and the Q5 High-Fidelity DNA Polymerase (NEB). Manufacturer's manuals were followed for reverse transcription and PCR. The full length and circularized CVB3-EGFP RNAs were used as templates for reserve transcription using the CVB3-Fluci/Spike/Cas9-vR® as the reverse primer. Then reverse transcription products were subject to PCR using the forward primer CVB3-EGFP-gF (F1) or CVB3-Fluci-vF (F2).

### Native Agarose Gel

100 ml of 0.8%-3.0% agarose gels were prepared in 1X TBE (89 mM Tris, 89 mM Boric acid, 3 mM EDTA) in the presence of 10 μl SYBR Safe (Thermo Fisher). Gels were operated horizontally in an Owl Easy Cast B2 Mini Gel Electrophoresis Systems (Thermo Scientific) at constantly 25W at room temperature with 1X TBE as running buffer for 70-120 min. For sample loading, each RNA sample (~500 ng) was mixed with equal volume of formamide loading buffer (Thermo Fisher) and denatured at 95° C. for 2 min. Formaldehyde agarose gels and urea polyacrylamide gels described below used identical sample loading procedure. Gels (including gels described below) were all imaged by a Bio-Rad Chemidoc XRS+ Imaging System.

### Formaldehyde Agarose Gel

0.8% and 1.5% formaldehyde agarose gels were prepared as previously described (Rio, D C et al, (2015)). Of note, we mixed SYBR Safe with gels and run at constantly 8W at 4° C. for 90 min.

### Urea Agarose Gel

For horizontal urea agarose gels, the horizontal Owl Easy Cast B1 Mini Gel Electrophoresis Systems (Thermo Scientific) was used. To prepare a 1.5% agarose gel with 6M urea, 0.75g agarose was firstly boiled in 40 ml DEPC H₂O and mixed with 18g urea. Then add 5 ml 10X TBE and 5 ul SYBR Green and top up with DEPC H₂O to 50 ml. Then pour the gel into the B1 Mini Gel cassette and allow gelation at 4° C. for several hours. For sample loading, each RNA sample (~500 ng) was mixed with equal volume of urea loading buffer (NEB) and denatured at 95° C. for 2 min. Gels were run at room temperature at 15W for 40 min.

For vertical urea agarose gels, the Bio-Rad Mini-PROTEAN Tetra Vertical Electrophoresis Cell system was used. Agarose was firstly boiled in DEPC H₂O, mixed with various amount of urea, top up with 10X TBE and DEPC H₂O and then poured into gel plates with 1.5 mm spacers. To avoid gels from slipping during running, a 0.75 mm stopper was stuck to the bottom edge of the bottom plate. Gels were placed at 4° C. for gelation for several hours. Combs need to be exposed by slipping down the cover plate before removing, since pulling out combs directly would damage gels. At this moment, clean gel wells with pipette tips and then slip back the cover plate. For the 4% agarose gel with 6M urea, gel plates were preheated to help avoid gelation during pouring the gel. Gels were run at room temperature at 15-25W for 15-30 min. For sample loading, each RNA sample (~100 ng) was mixed with equal volume of urea loading buffer (NEB) and denatured at 95° C. for 2 min. Formamide loading buffer will cause shadows of RNA bands and thus not suitable for urea agarose gels. Gels were stained in 10 ml 1X TBE with SYBR Green for 10 min before imaging.

### Example 1

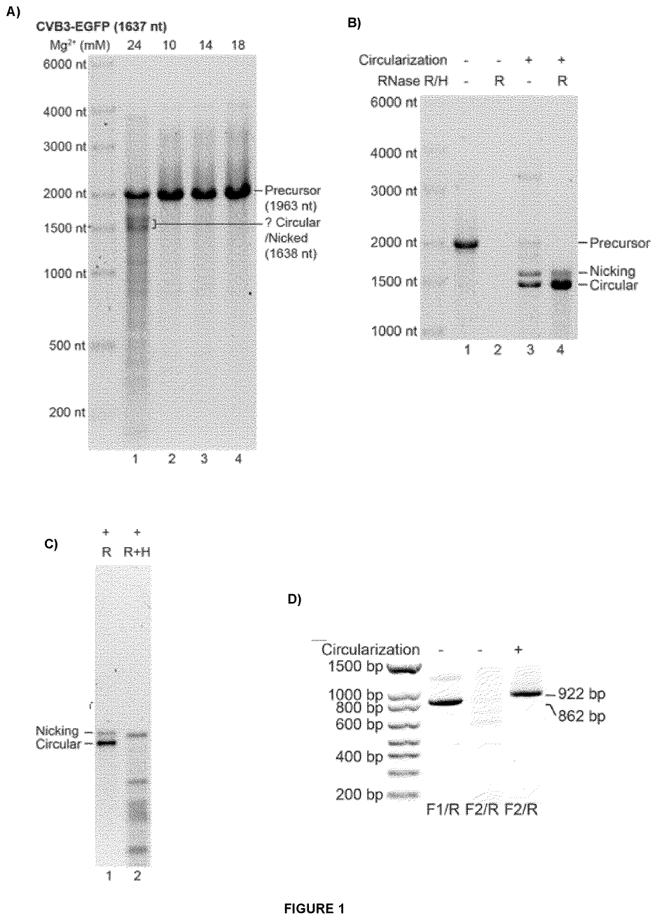

It has been reported that native agarose gel gives no separation between circular RNA and its linear counterpart (Zhang et al 2016). To test this, we cloned the CVB3-EGFP into the PIE construct. We firstly checked the in vitro transcribed RNA by a 0.8% native agarose gel run at constant 25W for 70 min (FIG. 1A, lane 1). Due to co-transcriptional splicing, multiple RNA species are already visible. Among them the full-length precursor (1963 nucleotides) can be easily identified but not the circular/nicked RNA (1638 nucleotides), as there are two dominant bands around the size of circular/nicked RNA. To reduce the complexity of IVT product, we lowered the concentration of Mg²⁺ in IVT to supress co-transcriptional splicing of the ribozyme as previously described (Zaug A L et al, 1993). As a result, the IVT product is now mainly the full-length precursor (FIG. 1A, lanes 2-4).

A circularization reaction was carried out on the full-length precursor and the products loaded on a 0.8% native agarose gel (FIG. 1B, lane 3). The post-transcriptional circularization procedure gives much less RNA species. As above, there are still two bands near where the position of circular/nicked CVB3-EGFP. The lower band runs at the same position as a 1500 nucleotide maker and the upper band runs at ~1600nt. Thus, the upper band looks like the circular/nicked CVB3-EGFP (1638 nucleotide). We digested the full-length precursor and circularized sample with an exonuclease, RNase R, and found that only the lower band is resistant to RNase R digestion, indicating that the lower band rather than the upper band contains circular CVB3-EGFP (FIG. 1B, lanes 1-4).

Since the lower band runs faster than the position expected for linear CVB3-EGFP, we hypothesized that the lower band is circular while the upper band is nicked circular CVB3-EGFP. We purified the lower band by gel extraction followed by RNase R digestion (FIG. 1C, lane 1). The identity of circular CVB3-EGFP was further confirmed by RT-PCR. The presence of a 922 bp product from circularized CVB3-EGFP confirms the circularization. (FIG. 1D). Then we digested the circular CVB3-EGFP by RNase H guided by a 34 mer DNA oligo and found the linearized CVB3-EGFP now moves similarly as the upper band (FIG. 1C, lane 2), indicating that the upper band is nicked CVB3-EGFP. The small difference between nicked and RNase H linearized RNA is the result of RNase H digestion which removes additional nucleotides.

Overall, these results demonstrate that instead of co-migrating with its linear counterpart, circular CVB3-EGFP runs faster than its linear counterpart in a 0.8% native agarose gel, which is similar to the observation that in native condition, circular RNA has a larger retention time than its linear counterpart in the Sepax gel-filtration column (Wesselhoeft, R. A et al, 2018).

### Example 2

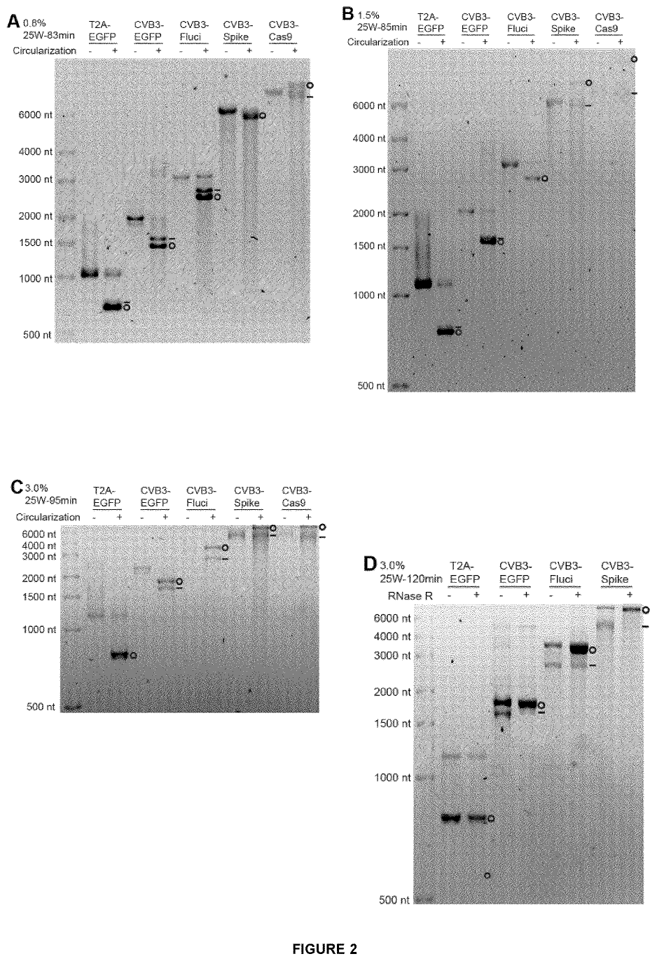

We made four other constructs that would produce circular EGFP (813 nucleotides), CVB3-Firefly luciferase (Fluci, 2600 nucleotides), CVB3-Spike protein of SARS Cov 2 fused with EGPF (Spike, 5468 nucleotides) and CVB3-spCas9 fused with EGFP (Cas9, 5756 nucleotides). We firstly did IVT and loaded these samples pre/post circularization for separation on a 0.8% agarose gel. As shown in FIG. 2A in this 0.8% gel, circular T2A-EGFP, CVB3-EGFP and CVB3-Fluci run faster than their nicked counterparts. Circular CVB3-Spike runs similarly as its nicked product and circular CVB3-Cas9 runs even slower than its full-length precursor. Consistently, circular and nicked CVB3-EGFP run as two bands (FIG. 2A). For circular EGFP and Fluci, we can also see two RNA species run faster than full-length precursor, with the lower band stronger than the upper band, indicating that circular RNAs run faster for these two RNAs. For Spike, we can see there are partly overlapped two bands below full-length precursor, but it's difficult to tell which one is circular RNA. But for the Cas9, a new band emerges upper of the precursor (FIG. 2A). We assume that the upper most band in Cas9 is the circular RNA (FIG. 2A). We loaded samples on a 1.5% and a 3% agarose gel (FIG. 2 B-C). As shown in FIG. 2B in the 1.5% gel, circular T2A-EGFP and CVB3-EGFP run faster than their linear counterparts, but the separation is much smaller, whilst circular CVB3-Fluci runs similarly as its linear counterpart. In the 1.5% gel, the separation between circular and nicked RNA for EGFP and CVB3-EGFP became smaller while for the Fluci, circular RNA migrates slightly slower than the nicked RNA. Circular CVB3-Spike now runs much slower than its precursor and the separation between circular CVB3-Cas9 and its linear precursor is larger. For Cas9, the upper band moves further away from the precursor. Interestingly, a new band emerges up of the precursor of the Spike. As shown in FIG. 2(C) in the 3% gel, circular RNAs all moves slower than their linear counterparts.

To further confirm these upper bands are circular RNAs, we did RNase R digestion and loaded these samples to a 3% agarose gel (FIG. 2D). It clearly shows that only the upper bands are resistant to RNase R digestion. The gel in FIG. 2D confirms that those upper bands are circular RNAs. These results demonstrate that separation between circular RNAs and its linear counterparts can be modulated by changing concentration of agarose in native agarose gel system.

### Example 3

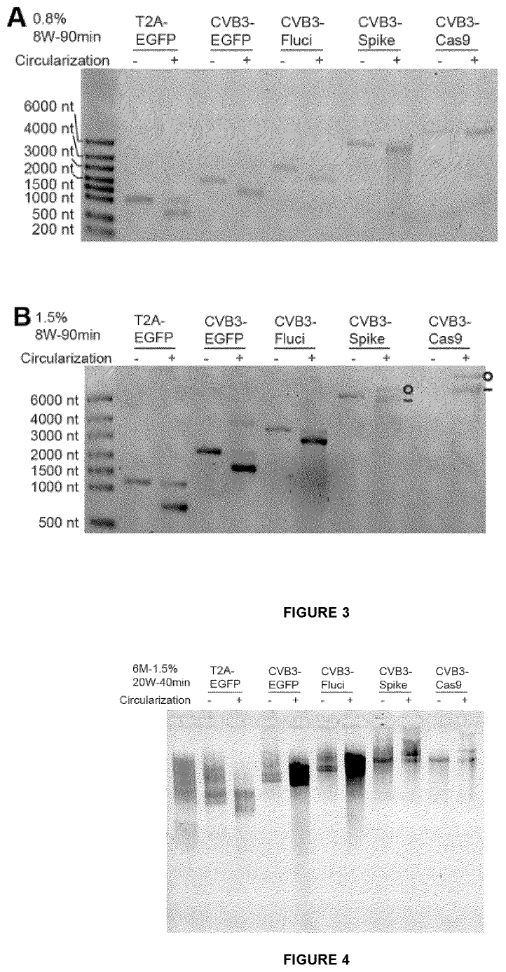

To test how circular RNA behaves in popular denaturing agarose gels which use formaldehyde as denaturing reagent we firstly loaded these samples to a 0.8% formaldehyde-agarose gel and found that there is no separation between circular RNA and their linear counterparts for all of those five RNAs (FIG. 3A). We then loaded these samples on to a 1.5% formaldehyde-agarose gel and found circular Spike and Cas9 do migrate slower than their linear counterparts (FIG. 3B), which is similar to the case in native agarose gels. But we were not able to see any separation between circRNAs and their linear counterparts for EGFP, CVB3-EGFP and Fluc. We hypothesised that once we load these samples to a higher concentration agarose gel, say 3%, we should be able to see separation for those three short RNAs. But 3% formaldehyde-agarose gel is difficult to handle. These results suggest that in formaldehyde-agarose gel, circular RNAs behave similarly as in native agarose gel.

### Example 4

Due to the toxicity of the formaldehyde, urea is sometimes used as a replacement (Rosen, J. M. et al (1975), Reijnders L et al (1973)). However, it is generally believed that urea is not a good denaturing reagent in agarose gel system for nucleic acids (Masek T et al (2005)). To test the ability of urea as a denaturing in agarose gels we loaded samples on to a 6M-1.5% urea-agarose gel in a horizontal electrophoresis system. As shown in FIG. 4, separation between circular RNAs and their linear counterparts was less clear using the horizontal electrophoresis system tested herewith a urea agarose gel.

We also used a vertical gel electrophoresis system, which is originally designed for protein PAGE.

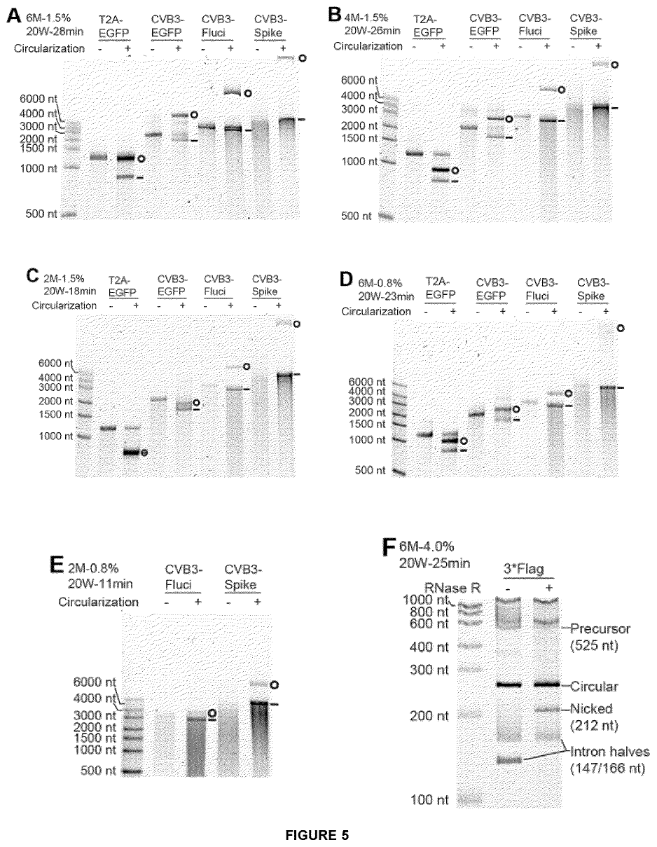

As shown in FIG. 5A, in a 6M-1.5% urea-agarose gel, instead of running faster than their linear counterparts, all circular RNA species migrate much more slowly than their linear counterparts. For example, the circular EGFP runs at the same position of full-length precursor and the rest of the circular RNAs all run slower than full length precursors (FIG. 5A).

The concentration of agarose was fixed at 1.5% and the concentration of urea was reduced to 4 M and 2 M. Separation between circular RNAs and their linear counterparts also reduces (FIG. 5B-C). For example, when urea is 4M, circular EGFP runs slower than its linear counterpart but faster than full-length precursor. While when urea is 2 M, circular EGFP co-migrates with its linear counterpart. These results clearly show that by adding urea to agarose gel system, circular RNAs can be separated from their linear counterparts. Considering that circular RNAs behave similarly in native agarose gels and denaturing formaldehyde gels, the migration differences between circular RNAs and their linear counterparts in denaturing urea-agarose gels should be attributed to urea, rather than denaturation of RNAs.

To test whether the concentration of agarose would influence separation between circular RNA and its linear counterpart in the urea-agarose gel system we loaded these samples on 0.8% agarose gel with 6 M urea, we can see that separation between circular RNAs and their linear counterparts becomes smaller when comparing to 1.5% agarose gel (FIG. 5D). Further lower urea concentration in 0.8% agarose gel makes the separation even smaller (FIG. 5E).

Accordingly, for a given concentration of agarose, the higher the concentration of urea, the larger the separation between circular RNAs and their linear counterparts. For given concentration of urea, the higher the concentration of agarose, the larger the separation between circular RNAs and their linear counterparts. Moreover, it can also be seen that for a given urea agarose gel, the longer the circular RNA, the larger the separation between circular RNAs and their linear counterparts. Therefore, these result show that it is better to resolve long circular RNAs in low concentration urea agarose gels and vice versa.

These results show that circular RNAs within the range of ~800-6000 nucleotide can be easily separated with proper concentration of agarose and urea.

To test whether this system works for short circular RNAs, we constructed a new vector which would produce a 212 nucleotides circular 3*Flag RNA. We loaded circularized samples to a 4% agarose gel with 6 M urea and found that there is a good separation between circular 3*Flag and its linear counterparts (FIG. 5F). The urea agarose gel system can also resolve short circular RNA.

Therefore, these results that circular RNA in the range of about 200 to about 6000 nucleotides can be separated from their linear counterpart in an agarose gel, using urea as the denaturing agent.

### Example 5

To test how gel running parameters influences the separation between circular RNAs and their linear counterparts, we tested different combination of running times and running power.

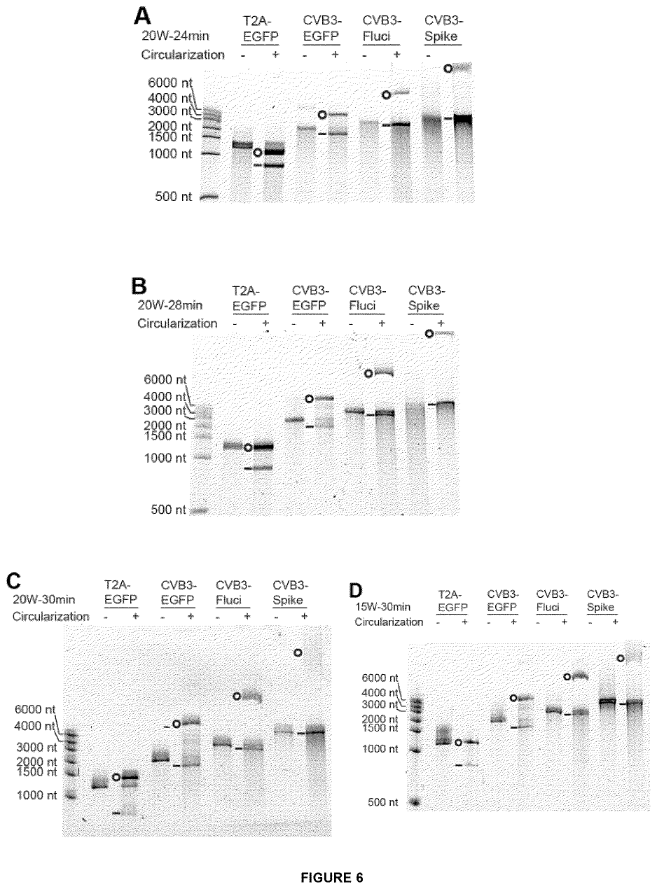

Using the 1.5% agarose gel with 6 M urea, we found that the longer running time or the higher the running power, the larger the separation between circular RNAs and their linear counterparts (FIG. 6).

As shown in FIGS. 6A-6B at a fixed running power the longer the running the larger the separation between circular RNAs and their linear counterparts. As shown in FIGS. 6C-6D at a fixed running the higher the running power the larger the separation between circular RNAs and their linear counterparts. For the gels in FIGS. 6C and 6D a denaturing formamide loading buffer was used. Formamide loading buffer causes shadows of RNA bands. For example, each species of the RNA maker has an upper shadow band.

These results demonstrate that separation between circular RNAs and their linear counterparts in the range of ~200-6000 nucleotides can be tuned by changing of urea/agarose concentration and running parameters.

The examples provided above shown that agarose gels using urea as the denaturing agent are shown to effectively separate circular RNA from its linear counterparts in samples. These results exemplify a new technique for identifying circular RNAs up to 6000 nucleotides from their linear counterparts in RNA containing samples. The invention provides a simple method for circular RNA identification and could help the development of better circular RNAs analysis strategies for circular RNA-based therapeutics.

Although the present disclosure has been described in detail with reference to the specific features, it will be apparent to those skilled in the art that this description is only for a preferred embodiment and does not limit the scope of the present disclosure.

### Sequences

Sequences referred to herein are set out in the following table:

| SEQ | | |
|---|---|---|
| ID | | |
| NO | Description | Sequence |
| 1 | pUCIDT- | TAATACGACTCACTATAGGGAGACCCTCGACCGTCGATTGTCCACTGGTCAACAATAGATGAC |
| | PIE- | TTACAACTAATCGGAAGGTGCAGAGACTCGACGGGAGCTACCCTAACGTCAAGACGAGGGTA |
| | Ana3.0- | AAGAGAGAGTCCAATTCTCAAAGCCAATAGGCAGTAGCGAAAGCTGCAAGAGAATGAAAATC |
| | empty | CGTTGACCTTAAACGGTCGTGTGGGTTCAAGTCCCTCCACCCCCAAGACGCTACGGACTTAA |
| | | ATAATTGAGCCTTAAAGAAGAAATTCTTTAAGTGGATGCTCTCAAACTCAGGGAAACCTAAATC |
| | | TAGTTATAGACAAGGCAATCCTGAGCCAAGCCGAAGTAGTAATTAGTAAGACCAGTGGACAAT |
| | | CGACGGATAACAGCATATCTAGCGGCCGCGATATC |
| 2 | pUCIDT- | TAATACGACTCACTATAGGGAGACCCTCGACCGTCGATTGTCCACTGGTCAACAATAGATGAC |
| | PIE- | TTACAACTAATCGGAAGGTGCAGAGACTCGACGGGAGCTACCCTAACGTCAAGACGAGGGTA |
| | Aan3.0- | AAGAGAGAGTCCAATTCTCAAAGCCAATAGGCAGTAGCGAAAGCTGCAAGAGAATGAAAATC |
| | CVB3- | CGTTGACCTTAAACGGTCGTGTGGGTTCAAGTCCCTCCACCCCCACGCCGGAAACGCAATAG |
| | EGFP | CCGAAAAACAAAAAACAAAAAAAACAAAAAAAAAACCAAAAAAAAAAACACATTAAAACAGCC |
| | | TGTGGGTTGATCCCACCCACAGGCCCATTGGGCGCTAGCACTCTGGTATCACGGTACCTTTG |
| | | TGCGCCTGTTTTATACCCCCTCCCCCAACTGTAACTTAGAAGTAACACACACCGATCAACAGT |
| | | CAGCGTGGCACACCAGCCACGTTTTGATCAAGCACTTCTGTTACCCCGGACTGAGTATCAAT |
| | | AGACTGCTCACGCGGTTGAAGGAGAAAGCGTTCGTTATCCGGCCAACTACTTCGAAAAACCT |
| | | AGTAACACCGTGGAAGTTGCAGAGTGTTTCGCTCAGCACTACCCCAGTGTAGATCAGGTCGA |
| | | TGAGTCACCGCATTCCCCACGGGCGACCGTGGCGGTGGCTGCGTTGGCGGCCTGCCCATG |
| | | GGGAAACCCATGGGACGCTCTAATACAGACATGGTGCGAAGAGTCTATTGAGCTAGTTGGTA |
| | | GTCCTCCGGCCCCTGAATGCGGCTAATCCTAACTGCGGAGCACACACCCTCAAGCCAGAGG |
| | | GCAGTGTGTCGTAACGGGCAACTCTGCAGCGGAACCGACTACTTTGGGTGTCCGTGTTTCAT |
| | | TTTATTCCTATACTGGCTGCTTATGGTGACAATTGAGAGATCGTTACCATATAGCTATTGGATT |
| | | GGCCATCCGGTGACTAATAGAGCTATTATATATCCCTTTGTTGGGTTTATACCACTTAGCTTGA |
| | | AAGAGGTTAAAACATTACAATTCATTGTTAAGTTGAATACAGCAAAATGGTGAGCAAGGGCGA |
| | | GGAGCTGTTCACCGGGGTGGTGCCCATCCTGGTCGAGCTGGACGGCGACGTAAACGGCCA |
| | | CAAGTTCAGCGTGTCCGGCGAGGGCGAGGGCGATGCCACCTACGGCAAGCTGACCCTGAA |
| | | GTTCATCTGCACCACCGGCAAGCTGCCCGTGCCCTGGCCCACCCTCGTGACCACCCTGACC |
| | | TACGGCGTGCAGTGCTTCAGCCGCTACCCCGACCACATGAAGCAGCACGACTTCTTCAAGTC |
| | | CGCCATGCCCGAAGGCTACGTCCAGGAGCGCACCATCTTCTTCAAGGACGACGGCAACTAC |
| | | AAGACCCGCGCCGAGGTGAAGTTCGAGGGCGACACCCTGGTGAACCGCATCGAGCTGAAG |
| | | GGCATCGACTTCAAGGAGGACGGCAACATCCTGGGGCACAAGCTGGAGTACAACTACAACA |
| | | GCCACAACGTCTATATCATGGCCGACAAGCAGAAGAACGGCATCAAGGTGAACTTCAAGATC |
| | | CGCCACAACATCGAGGACGGCAGCGTGCAGCTCGCCGACCACTACCAGCAGAACACCCCCA |
| | | TCGGCGACGGCCCCGTGCTGCTGCCCGACAACCACTACCTGAGCACCCAGTCCGCCCTGAG |
| | | CAAAGACCCCAACGAGAAGCGCGATCACATGGTCCTGCTGGAGTTCGTGACCGCCGCCGGG |
| | | ATCACTCTCGGCATGGACGAGCTGTACAAGTAAAAAAAACAAAAAACAAAACGGCTATTATGC |
| | | GTTACCGGCGAGACGCTACGGACTTAAATAATTGAGCCTTAAAGAAGAAATTCTTTAAGTGGA |
| | | TGCTCTCAAACTCAGGGAAACCTAAATCTAGTTATAGACAAGGCAATCCTGAGCCAAGCCGAA |
| | | GTAGTAATTAGTAAGACCAGTGGACAATCGACGGATAACAGCATATCTAGCGGCCGCGATAT |
| | | C |
| 3 | pUCIDT- | TAATACGACTCACTATAGGGAGACCCTCGACCGTCGATTGTCCACTGGTCAACAATAGATGAC |
| | PIE- | TTACAACTAATCGGAAGGTGCAGAGACTCGACGGGAGCTACCCTAACGTCAAGACGAGGGTA |
| | Aan3.0- | AAGAGAGAGTCCAATTCTCAAAGCCAATAGGCAGTAGCGAAAGCTGCAAGAGAATGAAAATC |
| | CVB3-Fluci | CGTTGACCTTAAACGGTCGTGTGGGTTCAAGTCCCTCCACCCCCACGCCGGAAACGCAATAG |
| | | CCGAAAAACAAAAAACAAAAAAAACAAAAAAAAAACCAAAAAAACAAAACACATTAAAACAGCC |
| | | TGTGGGTTGATCCCACCCACAGGCCCATTGGGCGCTAGCACTCTGGTATCACGGTACCTTTG |
| | | TGCGCCTGTTTTATACCCCCTCCCCCAACTGTAACTTAGAAGTAACACACACCGATCAACAGT |
| | | CAGCGTGGCACACCAGCCACGTTTTGATCAAGCACTTCTGTTACCCCGGACTGAGTATCAAT |
| | | AGACTGCTCACGCGGTTGAAGGAGAAAGCGTTCGTTATCCGGCCAACTACTTCGAAAAACCT |
| | | AGTAACACCGTGGAAGTTGCAGAGTGTTTCGCTCAGCACTACCCCAGTGTAGATCAGGTCGA |
| | | TGAGTCACCGCATTCCCCACGGGCGACCGTGGCGGTGGCTGCGTTGGCGGCCTGCCCATG |
| | | GGGAAACCCATGGGACGCTCTAATACAGACATGGTGCGAAGAGTCTATTGAGCTAGTTGGTA |
| | | GTCCTCCGGCCCCTGAATGCGGCTAATCCTAACTGCGGAGCACACACCCTCAAGCCAGAGG |
| | | GCAGTGTGTCGTAACGGGCAACTCTGCAGCGGAACCGACTACTTTGGGTGTCCGTGTTTCAT |
| | | TTTATTCCTATACTGGCTGCTTATGGTGACAATTGAGAGATCGTTACCATATAGCTATTGGATT |
| | | GGCCATCCGGTGACTAATAGAGCTATTATATATCCCTTTGTTGGGTTTATACCACTTAGCTTGA |
| | | AAGAGGTTAAAACATTACAATTCATTGTTAAGTTGAATACAGCAAAATGGAAGATGCCAAAAAC |
| | | ATTAAGAAGGGCCCAGCGCCATTCTACCCACTCGAAGACGGTACCGCCGGCGAGCAGCTGC |
| | | ACAAAGCCATGAAGCGCTACGCCCTGGTGCCCGGCACCATCGCCTTTACCGACGCACATATC |
| | | GAGGTGGACATTACCTACGCCGAGTACTTCGAGATGAGCGTTCGGCTGGCAGAAGCTATGAA |
| | | GCGCTATGGGCTGAATACAAACCATCGGATCGTGGTGTGCAGCGAGAATAGCTTGCAGTTCT |
| | | TCATGCCCGTGTTGGGTGCCCTGTTCATCGGTGTGGCTGTGGCCCCAGCTAACGACATCTAC |
| | | AACGAGCGCGAGCTGCTGAACAGCATGGGCATCAGCCAGCCCACCGTCGTATTCGTGAGCA |
| | | AGAAAGGGCTGCAAAAGATCCTCAACGTGCAAAAGAAGCTACCGATCATACAAAAGATCATCA |
| | | TCATGGATAGCAAGACCGACTACCAGGGCTTCCAAAGCATGTACACCTTCGTGACTTCCCATT |
| | | TGCCACCCGGCTTCAACGAGTACGACTTCGTGCCCGAGAGCTTCGACCGGGACAAAACCAT |
| | | CGCCCTGATCATGAACAGTAGTGGCAGTACCGGATTGCCCAAGGGCGTAGCCCTACCGCAC |
| | | CGCACCGCTTGTGTCCGATTCAGTCATGCCCGCGACCCCATCTTCGGCAACCAGATCATCCC |
| | | CGACACCGCTATCCTCAGCGTGGTGCCATTTCACCACGGCTTCGGCATGTTCACCACGCTGG |
| | | GCTACTTGATCTGCGGCTTTCGGGTCGTGCTCATGTACCGCTTCGAGGAGGAGCTATTCTTG |
| | | CGCAGCTTGCAAGACTATAAGATTCAATCTGCCCTGCTGGTGCCCACACTATTTAGCTTCTTC |
| | | GCTAAGAGCACCCTCATTGATAAGTACGACCTAAGCAACTTGCACGAGATCGCCAGCGGCGG |
| | | GGCGCCGCTCAGCAAGGAGGTAGGTGAGGCCGTGGCTAAACGCTTCCACCTACCAGGCATC |
| | | CGCCAGGGCTACGGCCTGACAGAAACAACCAGCGCCATTCTGATCACCCCCGAAGGGGACG |
| | | ACAAGCCTGGCGCAGTAGGCAAGGTGGTGCCCTTCTTCGAGGCTAAGGTGGTGGACTTGGA |
| | | CACCGGCAAGACACTGGGTGTGAACCAGCGCGGCGAGCTGTGCGTCCGTGGCCCCATGATC |
| | | ATGAGCGGCTACGTTAACAACCCCGAGGCTACAAACGCACTCATCGACAAGGACGGCTGGC |
| | | TGCACAGCGGCGACATCGCCTACTGGGACGAGGACGAGCACTTCTTCATCGTGGACCGGCT |
| | | GAAGAGCCTGATCAAATACAAGGGCTACCAGGTAGCCCCAGCCGAACTGGAGAGCATCCTG |
| | | CTGCAACACCCCAACATCTTCGACGCCGGGGTCGCCGGCCTGCCCGACGACGATGCCGGC |
| | | GAGCTGCCCGCCGCAGTCGTCGTGCTGGAACACGGTAAAACCATGACCGAGAAGGAGATCG |
| | | TGGACTATGTGGCCAGCCAGGTTACAACCGCCAAGAAGCTGCGCGGTGGTGTTGTGTTCGT |
| | | GGACGAGGTGCCTAAAGGACTGACCGGTAAGTTGGACGCCCGCAAGATCCGCGAGATTCTC |
| | | ATTAAGGCCAAGAAGGGCGGCAAGATCGCCGTGAGCAGTGATTACAAGGATGATGATGATAA |
| | | GTAAAAAAAACAAAAAAAAAACGGCTATTATGCGTTACCGGCGAGACGCTACGGACTTAAAT |
| | | AATTGAGCCTTAAAGAAGAAATTCTTTAAGTGGATGCTCTCAAACTCAGGGAAACCTAAATCTA |
| | | GTTATAGACAAGGCAATCCTGAGCCAAGCCGAAGTAGTAATTAGTAAGACCAGTGGACAATC |
| | | GACGGATAACAGCATATCTAGCGGCCGCGATATC |
| 4 | pUCIDT- | TAATACGACTCACTATAGGGAGACCCTCGACCGTCGATTGTCCACTGGTCAACAATAGATGAC |
| | PIE- | TTACAACTAATCGGAAGGTGCAGAGACTCGACGGGAGCTACCCTAACGTCAAGACGAGGGTA |
| | Aan3.0- | AAGAGAGAGTCCAATTCTCAAAGCCAATAGGCAGTAGCGAAAGCTGCAAGAGAATGAAAATC |
| | CVB3- | CGTTGACCTTAAACGGTCGTGTGGGTTCAAGTCCCTCCACCCCCACGCCGGAAACGCAATAG |
| | Spike- | CCGAAAAACAAAAAACAAAAAAAAAAAAAAAAAACCAAAAAAACAAAACACATTAAAACAGCC |
| | EGFP | TGTGGGTTGATCCCACCCACAGGCCCATTGGGCGCTAGCACTCTGGTATCACGGTACCTTTG |
| | | TGCGCCTGTTTTATACCCCCTCCCCCAACTGTAACTTAGAAGTAACACACACCGATCAACAGT |
| | | CAGCGTGGCACACCAGCCACGTTTTGATCAAGCACTTCTGTTACCCCGGACTGAGTATCAAT |
| | | AGACTGCTCACGCGGTTGAAGGAGAAAGCGTTCGTTATCCGGCCAACTACTTCGAAAAACCT |
| | | AGTAACACCGTGGAAGTTGCAGAGTGTTTCGCTCAGCACTACCCCAGTGTAGATCAGGTCGA |
| | | TGAGTCACCGCATTCCCCACGGGCGACCGTGGCGGTGGCTGCGTTGGCGGCCTGCCCATG |
| | | GGGAAACCCATGGGACGCTCTAATACAGACATGGTGCGAAGAGTCTATTGAGCTAGTTGGTA |
| | | GTCCTCCGGCCCCTGAATGCGGCTAATCCTAACTGCGGAGCACACACCCTCAAGCCAGAGG |
| | | GCAGTGTGTCGTAACGGGCAACTCTGCAGCGGAACCGACTACTTTGGGTGTCCGTGTTTCAT |
| | | TTTATTCCTATACTGGCTGCTTATGGTGACAATTGAGAGATCGTTACCATATAGCTATTGGATT |
| | | GGCCATCCGGTGACTAATAGAGCTATTATATATCCCTTTGTTGGGTTTATACCACTTAGCTTGA |
| | | AAGAGGTTAAAACATTACAATTCATTGTTAAGTTGAATACAGCAAAATGGGGATTCTGCCTAGC |
| | | CCCGGCATGCCCGCCCTCCTGAGCCTGGTGAGCCTGCTGAGCGTCCTGTTGATGGGGTGCG |
| | | TGGCCGAGACCGGCACACAGTGCGTGAACCTGACAACTAGAACACAGCTGCCCCCAGCCTA |
| | | CACCAACAGCTTTACTAGGGGAGTGTACTACCCTGATAAGTGCTTCAGGAGCAGTGTGCTGC |
| | | ACAGCACCCAGGACCTCTTTTTGCCCTTTTTCAGTAACGTGACCTGGTTCCACGCTATCCACG |
| | | TGAGCGGCACAAACGGCACAAAACGGTTCGATAACCCAGTGCTGCCCTTCAACGACGGCGT |
| | | CTACTTCGCCAGCACTGAGAAGTCCAACATTATCAGGGGGTGGATCTTCGGGACAACCCTGG |
| | | ACAGCAAGACACAGAGCCTCCTGATTGTGAACAACGCCACCAACGTGGTCATTAAGGTCTGC |
| | | GAGTTCCAGTTCTGCAACGACCCCTTTCTGGGGGTGTACTACCACAAGAACAACAAGTCCTG |
| | | GATGGAGTCCGAGTTTCGGGTGTACTCCAGCGCCAACAACTGCACATTTGAGTACGTGAGCC |
| | | AGCCCTTCTTGATGGATCTGGAGGGCAAGCAGGGGAACTTTAAGAACCTTCGGGAGTTCGTC |
| | | TTTAAGAACATCGACGGGTACTTTAAGATCTACAGCAAGCACACCCCTATTAACCTGGTCCGC |
| | | GATCTGCCCCAGGGCTTCAGCGCCCTGGAGCCACTGGTGGACCTCCCCATCGGGATCAACA |
| | | TTACTAGGTTCCAGACACTCCTGGCCCTGCACCGGAGCTACCTGACTCCCGGCGATAGCTCC |
| | | AGCGGGTGGACTGCCGGGGCCGCCGCCTACTACGTCGGCTACCTGCAGCCCAGGACATTC |
| | | CTGCTCAAGTACAACGAGAACGGCACCATCACTGATGCCGTGGACTGCGCTCTCGATCCCCT |
| | | GAGCGAGACAAAGTGCACCCTGAAGTCCTTCACCGTGGAGAAGGGGATCTACCAGACATCTA |
| | | ACTTCAGGGTGCAGCCCACTGAGTCCATTGTGAGATTTCCTAACATCACCAACCTGTGCCCCT |
| | | TCGGCGAGGTCTTTAACGCCACACGGTTCGCCAGCGTCTACGCTTGGAACAGGAAGCGGAT |
| | | CTCCAACTGCGTGGCCGACTACAGCGTGCTGTACAACAGCGCCTCCTTCAGCACCTTTAAGT |
| | | GCTACGGCGTGAGCCCCACTAAGCTGAACGACCTGTGCTTCACTAACGTGTACGCTGATAGC |
| | | TTCGTGATTAGGGGCGATGAGGTCCGGCAGATTGCCCCCGGCCAGACAGGGAAGATCGCTG |
| | | ACTACAACTATAAGCTGCCCGATGACTTCACAGGGTGTGTGATCGCCTGGAACAGCAACAAC |
| | | CTGGATAGCAAGGTCGGGGGGAACTACAACTACCTGTACAGGCTGTTCAGAAAGTCCAACCT |
| | | GAAGCCCTTCGAGCGGGATATCAGCACTGAGATCTACCAGGCCGGGAGCACCCCCTGCAAC |
| | | GGCGTCGAGGGCTTTAACTGCTACTTTCCCCTCCAGAGCTACGGCTTCCAACCCACCAACGG |
| | | CGTGGGGTACCAGCCCTACCGGGTCGTGGTGCTTAGCTTCGAGCTCCTGCACGCCCCTGCA |
| | | ACCGTGTGCGGCCCCAAGAAGAGCACAAACCTCGTGAAGAACAAGTGCGTGAACTTCAACTT |
| | | CAACGGGCTGACCGGCACCGGCGTGCTGACTGAGAGTAACAAGAAGTTTCTCCCCTTCCAG |
| | | CAGTGCGGGAGGGATATTGCCGACACTACTGATGCCGTGCGCGACCCTCAGACCCTTGAGA |
| | | TTCTGGACATCACTCCCTGCTCCTTCGGCGGAGTGTCCGTGATCACACCAGGGACCAACACT |
| | | AGCAACCAGGTCGCCGTCTTGTACCAGGACGTGAACTGCACCGAGGTGCCCGTGGCCATCC |
| | | ACGCCGATCAGCTGACCCCAACTTGGCGGGTGTACTCCACCGGGAGCAACGTGTTTCAGAC |
| | | ACGGGCCGGGTGCCTGATCGGGGCTGAGCATGTCAACAACAGCTACGAGTGCGATATTCCT |
| | | ATCGGCGCCGGGATTTGCGCCAGCTACCAGACACAGACAAACAGCCCTGGCTCGGCCAGCT |
| | | CCGTCGCCAGCCAGAGCATCATCGCCTACACAATGTCCTTGGGGGCCGAGAACAGCGTCGC |
| | | CTACAGCAACAACAGCATTGCCATCCCCACCAACTTCACAATCAGCGTCACAACCGAGATCCT |
| | | GCCCGTGAGCATGACTAAGACATCCGTGGATTGCACAATGTACATCTGCGGCGACTCCACAG |
| | | AGTGCAGTAACCTGCTGCTCCAGTACGGGTCCTTTTGTACCCAGCTGAACAGAGCCTTGACC |
| | | GGCATTGCCGTGGAGCAGGACAAGAACACCCAGGAGGTGTTTGCCCAGGTGAAGCAGATCT |
| | | ACAAGACACCCCCCATAAAGGACTTCGGGGGCTTTAACTTCTCCCAGATCTTGCCCGACCCC |
| | | AGCAAGCCCTCCAAGCGGTCATTCATTGAGGACCTGCTGTTTAACAAGGTGACCCTCGCTGA |
| | | TGCAGGGTTTATCAAGCAGTACGGCGATTGCCTGGGGGACATTGCCGCTAGGGACTTGATCT |
| | | GCGCCCAGAAGTTCAACGGCCTGACCGTGCTCCCCCCCCTGTTGACAGACGAGATGATCGC |
| | | CCAGTACACTAGCGCTCTGCTGGCCGGCACTATCACCAGCGGCTGGACCTTTGGGGCCGGC |
| | | GCCGCCCTCCAGATTCCATTCGCCATGCAGATGGCCTACCGCTTCAACGGCATCGGCGTGA |
| | | CTCAGAACGTGCTGTACGAGAACCAGAAGCTGATTGCCAACCAGTTCAACTCCGCCATCGGC |
| | | AAGATCCAGGACAGCCTGTCCAGCACAGCATCCGCCCTGGGGAAGTTGCAGGATGTGGTGA |
| | | ACCAGAACGCCCAGGCCCTGAACACACTGGTGAAGCAGCTGAGCAGCAACTTTGGCGCCAT |
| | | CAGTAGCGTCCTGAACGACATCCTGTCACGGCTTGATCCACCAGAGGCCGAGGTCCAGATC |
| | | GATCGGCTGATTACAGGCCGGCTGCAGAGTCTCCAGACCTACGTCACCCAGCAGCTGATCC |
| | | GGGCTGCCGAGATTAGAGCCTCCGCCAATCTCGCCGCCACAAAGATGAGTGAATGCGTCCT |
| | | GGGCCAGAGCAAAAGGGTCGACTTTTGCGGGAAGGGGTACCACCTGATGTCCTTTCCCCAG |
| | | TCCGCCCCTCACGGAGTGGTGTTCCTCCATGTCACATACGTGCCTGCCCAGGAGAAGAACTT |
| | | CACTACAGCCCCCGCCATTTGCCACGATGGGAAGGCCCATTTCCCTAGAGAGGGCGTGTTC |
| | | GTGTCCAACGGGACCCACTGGTTCGTGACTCAGCGGAACTTTTATGAACCCCAGATTATCAC |
| | | AACCGATAACACATTTGTCTCCGGCAACTGTGATGTGGTCATCGGCATCGTGAACAACACCG |
| | | TGTACGACCCACTGCAGCCTGAGCTTGACAGCTTTAAGGAGGAGCTGGACAAGTACTTTAAG |
| | | AACCACACCAGCCCTGATGTGGACCTTGGCGACATCAGCGGCATTAACGCCAGCGTGGTGA |
| | | ACATTCAGAAGGAGATCGATAGACTGAACGAGGTGGCCAAGAACTTGAACGAGTCACTGATT |
| | | GACCTGCAGGAGCTGGGGAAGTACGAGCAGTACATTAAGGGGTCCGGGAGGGAGAACCTGT |
| | | ACTTTCAGGGGGGGGGGGGGTCCGGCTACATTCCCGAGGCCCCAAGGGATGGCCAGGCCT |
| | | ACGTGAGGAAGGATGGGGAGTGGGTGCTGCTGAGCACCTTCTTGGGCAGCGGCAGCAGTAT |
| | | GGTGAGCAAGGGCGAGGAGCTGTTCACCGGGGTGGTGCCCATCCTGGTCGAGCTGGACGG |
| | | CGACGTAAACGGCCACAAGTTCAGCGTGTCCGGCGAGGGCGAGGGCGATGCCACCTACGG |
| | | CAAGCTGACCCTGAAGTTCATCTGCACCACCGGCAAGCTGCCCGTGCCCTGGCCCACCCTC |
| | | GTGACCACCCTGACCTACGGCGTGCAGTGCTTCAGCCGCTACCCCGACCACATGAAGCAGC |
| | | ACGACTTCTTCAAGTCCGCCATGCCCGAAGGCTACGTCCAGGAGCGCACCATCTTCTTCAAG |
| | | GACGACGGCAACTACAAGACCCGCGCCGAGGTGAAGTTCGAGGGCGACACCCTGGTGAACC |
| | | GCATCGAGCTGAAGGGCATCGACTTCAAGGAGGACGGCAACATCCTGGGGCACAAGCTGGA |
| | | GTACAACTACAACAGCCACAACGTCTATATCATGGCCGACAAGCAGAAGAACGGCATCAAGG |
| | | TGAACTTCAAGATCCGCCACAACATCGAGGACGGCAGCGTGCAGCTCGCCGACCACTACCA |
| | | GCAGAACACCCCCATCGGCGACGGCCCCGTGCTGCTGCCCGACAACCACTACCTGAGCACC |
| | | CAGTCCGCCCTGAGCAAAGACCCCAACGAGAAGCGCGATCACATGGTCCTGCTGGAGTTCG |
| | | TGACCGCCGCCGGGATCACTCTCGGCATGGACGAGCTGTACAAGTAAAAAAAACAAAAAACA |
| | | AAACGGCTATTATGCGTTACCGGCGAGACGCTACGGACTTAAATAATTGAGCCTTAAAGAAGA |
| | | AATTCTTTAAGTGGATGCTCTCAAACTCAGGGAAACCTAAATCTAGTTATAGACAAGGCAATC |
| | | CTGAGCCAAGCCGAAGTAGTAATTAGTAAGACCAGTGGACAATCGACGGATAACAGCATATC |
| | | TAGCGGCCGCGATATC |
| 5 | pUCIDT- | TAATACGACTCACTATAGGGAGACCCTCGACCGTCGATTGTCCACTGGTCAACAATAGATGAC |
| | PIE- | TTACAACTAATCGGAAGGTGCAGAGACTCGACGGGAGCTACCCTAACGTCAAGACGAGGGTA |
| | Aan3.0- | AAGAGAGAGTCCAATTCTCAAAGCCAATAGGCAGTAGCGAAAGCTGCAAGAGAATGAAAATC |
| | CVB3- | CGTTGACCTTAAACGGTCGTGTGGGTTCAAGTCCCTCCACCCCCACGCCGGAAACGCAATAG |
| | Cas9- | CCGAAAAACAAAAAACAAAAAAAACAAAAAAAAAACCAAAAAAACAAAACACATTAAAACAGCC |
| | EGFP | TGTGGGTTGATCCCACCCACAGGCCCATTGGGCGCTAGCACTCTGGTATCACGGTACCTTTG |
| | | TGCGCCTGTTTTATACCCCCTCCCCCAACTGTAACTTAGAAGTAACACACACCGATCAACAGT |
| | | CAGCGTGGCACACCAGCCACGTTTTGATCAAGCACTTCTGTTACCCCGGACTGAGTATCAAT |
| | | AGACTGCTCACGCGGTTGAAGGAGAAAGCGTTCGTTATCCGGCCAACTACTTCGAAAAACCT |
| | | AGTAACACCGTGGAAGTTGCAGAGTGTTTCGCTCAGCACTACCCCAGTGTAGATCAGGTCGA |
| | | TGAGTCACCGCATTCCCCACGGGCGACCGTGGCGGTGGCTGCGTTGGCGGCCTGCCCATG |
| | | GGGAAACCCATGGGACGCTCTAATACAGACATGGTGCGAAGAGTCTATTGAGCTAGTTGGTA |
| | | GTCCTCCGGCCCCTGAATGCGGCTAATCCTAACTGCGGAGCACACACCCTCAAGCCAGAGG |
| | | GCAGTGTGTCGTAACGGGCAACTCTGCAGCGGAACCGACTACTTTGGGTGTCCGTGTTTCAT |
| | | TTTATTCCTATACTGGCTGCTTATGGTGACAATTGAGAGATCGTTACCATATAGCTATTGGATT |
| | | GGCCATCCGGTGACTAATAGAGCTATTATATATCCCTTTGTTGGGTTTATACCACTTAGCTTGA |
| | | AAGAGGTTAAAACATTACAATTCATTGTTAAGTTGAATACAGCAAAATGGATAAGAAATACTCA |
| | | ATAGGCTTAGATATCGGCACAAATAGCGTCGGATGGGCGGTGATCACTGATGAATATAAGGT |
| | | TCCGTCTAAAAAGTTCAAGGTTCTGGGAAATACAGACCGCCACAGTATCAAAAAAAATCTTAT |
| | | AGGGGCTCTTTTATTTGACAGTGGAGAGACAGCGGAAGCGACTCGTCTCAAACGGACAGCTC |
| | | GTAGAAGGTATACACGTCGGAAGAATCGTATTTGTTATCTACAGGAGATTTTTTCAAATGAGAT |
| | | GGCGAAAGTAGATGATAGTTTCTTTCATCGACTTGAAGAGTCTTTTTTGGTGGAAGAAGACAA |
| | | GAAGCATGAACGTCATCCTATTTTTGGAAATATAGTAGATGAAGTTGCTTATCATGAGAAATAT |
| | | CCAACTATCTATCATCTGCGAAAAAAATTGGTAGATTCTACTGATAAAGCGGATTTGCGCTTAA |
| | | TCTATTTGGCCTTAGCGCATATGATTAAGTTTCGTGGTCATTTTTTGATTGAGGGAGATTTAAA |
| | | TCCTGATAATAGTGATGTGGACAAACTATTTATCCAGTTGGTACAAACCTACAATCAATTATTT |
| | | GAAGAAAACCCTATTAACGCAAGTGGAGTAGATGCTAAAGCGATTCTTTCTGCACGATTGAGT |
| | | AAATCAAGACGATTAGAAAATCTCATTGCTCAGCTCCCCGGTGAGAAGAAAAATGGCTTATTT |
| | | GGGAATCTCATTGCTTTGTCATTGGGTTTGACCCCTAATTTTAAATCAAATTTTGATTTGGCAG |
| | | AAGATGCTAAATTACAGCTTTCAAAAGATACTTACGATGATGATTTAGATAATTTATTGGCGCA |
| | | AATTGGAGATCAATATGCTGATTTGTTTTTGGCAGCTAAGAATTTATCAGATGCTATTTTACTTT |
| | | CAGATATCCTAAGAGTAAATACTGAAATAACTAAGGCTCCCCTATCAGCTTCAATGATTAAACG |
| | | CTACGATGAACATCATCAAGACTTGACTCTTTTAAAAGCTTTAGTTCGACAACAACTTCCAGAA |
| | | AAGTATAAAGAAATCTTTTTTGATCAATCAAAAAACGGATATGCAGGTTATATTGATGGGGGAG |
| | | CTAGCCAAGAAGAATTTTATAAATTTATCAAACCAATTTTAGAAAAAATGGATGGTACTGAGGA |
| | | ATTATTGGTGAAACTAAATCGTGAAGATTTGCTGCGCAAGCAACGGACCTTTGACAACGGCTC |
| | | TATTCCCCATCAAATTCACTTGGGTGAGCTGCATGCTATTTTGAGAAGACAAGAAGACTTTTAT |
| | | CCATTTTTAAAAGACAATCGTGAGAAGATTGAAAAAATCTTGACTTTTCGAATTCCTTATTATGT |
| | | TGGTCCATTGGCGCGTGGCAATAGTCGTTTTGCATGGATGACTCGGAAGTCTGAAGAAACAA |
| | | TTACCCCATGGAATTTTGAAGAAGTTGTCGATAAAGGTGCTTCAGCTCAATCATTTATTGAACG |
| | | CATGACAAACTTTGATAAAAATCTTCCAAATGAAAAAGTACTACCAAAACATAGTTTGCTTTAT |
| | | GAGTATTTTACGGTTTATAACGAATTGACAAAGGTCAAATATGTTACTGAAGGAATGCGAAAAC |
| | | CAGCATTTCTTTCAGGTGAACAGAAGAAAGCCATTGTTGATTTACTCTTCAAAACAAATCGAAA |
| | | AGTAACCGTTAAGCAATTAAAAGAAGATTATTTCAAAAAAATAGAATGTTTTGATAGTGTTGAA |
| | | ATTTCAGGAGTTGAAGATAGATTTAATGCTTCATTAGGTACCTACCATGATTTGCTAAAAATTA |
| | | TTAAAGATAAAGATTTTTTGGATAATGAAGAAAATGAAGATATCTTAGAGGATATTGTTTTAACA |
| | | TTGACCTTATTTGAAGATAGGGAGATGATTGAGGAAAGACTTAAAACATATGCTCACCTCTTTG |
| | | ATGATAAGGTGATGAAACAGCTTAAACGTCGCCGTTATACTGGTTGGGGACGTTTGTCTCGAA |
| | | AATTGATTAATGGTATTAGGGATAAGCAATCTGGCAAAACAATATTAGATTTTTTGAAATCAGA |
| | | TGGTTTTGCCAATCGCAATTTTATGCAGCTGATCCATGATGATAGTTTGACATTTAAAGAAGAC |
| | | ATTCAAAAAGCACAAGTGTCTGGACAAGGCGATAGTTTACATGAACATATTGCAAATTTAGCT |
| | | GGTAGCCCTGCTATTAAAAAAGGTATTTTACAGACTGTAAAAGTTGTTGATGAATTGGTCAAAG |
| | | TAATGGGGCGGCATAAGCCAGAAAATATCGTTATTGAAATGGCACGTGAAAATCAGACAACTC |
| | | AAAAGGGCCAGAAAAATTCGCGAGAGCGTATGAAACGAATCGAAGAAGGTATCAAAGAATTA |
| | | GGAAGTCAGATTCTTAAAGAGCATCCTGTTGAAAATACTCAATTGCAAAATGAAAAGCTCTATC |
| | | TCTATTATCTCCAAAATGGAAGAGACATGTATGTGGACCAAGAATTAGATATTAATCGTTTAAG |
| | | TGATTATGATGTCGATCACATTGTTCCACAAAGTTTCCTTAAAGACGATTCAATAGACAATAAG |
| | | GTCTTAACGCGTTCTGATAAAAATCGTGGTAAATCGGATAACGTTCCAAGTGAAGAAGTAGTC |
| | | AAAAAGATGAAAAACTATTGGAGACAACTTCTAAACGCCAAGTTAATCACTCAACGTAAGTTTG |
| | | ATAATTTAACGAAAGCTGAACGTGGAGGTTTGAGTGAACTTGATAAAGCTGGTTTTATCAAAC |
| | | GCCAATTGGTTGAAACTCGCCAAATCACTAAGCATGTGGCACAAATTTTGGATAGTCGCATGA |
| | | ATACTAAATACGATGAAAATGATAAACTTATTCGAGAGGTTAAAGTGATTACCTTAAAATCTAA |
| | | ATTAGTTTCTGACTTCCGAAAAGATTTCCAATTCTATAAAGTACGTGAGATTAACAATTACCAT |
| | | CATGCCCATGATGCGTATCTAAATGCCGTCGTTGGAACTGCTTTGATTAAGAAATATCCAAAA |
| | | CTTGAATCGGAGTTTGTCTATGGTGATTATAAAGTTTATGATGTTCGTAAAATGATTGCTAAGT |
| | | CTGAGCAAGAAATAGGCAAAGCAACCGCAAAATATTTCTTTTACTCTAATATCATGAACTTCTT |
| | | CAAAACAGAAATTACACTTGCAAATGGAGAGATTCGCAAACGCCCTCTAATCGAAACTAATGG |
| | | GGAAACTGGAGAAATTGTCTGGGATAAAGGGCGAGATTTTGCCACAGTGCGCAAAGTATTGT |
| | | CCATGCCCCAAGTCAATATTGTCAAGAAAACAGAAGTACAGACAGGCGGATTCTCCAAGGAG |
| | | TCAATTTTACCAAAAAGAAATTCGGACAAGCTTATTGCTCGTAAAAAAGACTGGGATCCAAAAA |
| | | AATATGGTGGTTTTGATAGTCCAACGGTAGCTTATTCAGTCCTAGTGGTTGCTAAGGTGGAAA |
| | | AAGGGAAATCGAAGAAGTTAAAATCCGTTAAAGAGTTACTAGGGATCACAATTATGGAAAGAA |
| | | GTTCCTTTGAAAAAAATCCGATTGACTTTTTAGAAGCTAAAGGATATAAGGAAGTTAAAAAAGA |
| | | CTTAATCATTAAACTACCTAAATATAGTCTTTTTGAGTTAGAAAACGGTCGTAAACGGATGCTG |
| | | GCTAGTGCCGGAGAATTACAAAAAGGAAATGAGCTGGCTCTGCCAAGCAAATATGTGAATTTT |
| | | TTATATTTAGCTAGTCATTATGAAAAGTTGAAGGGTAGTCCAGAAGATAACGAACAAAAACAAT |
| | | TGTTTGTGGAGCAGCATAAGCATTATTTAGATGAGATTATTGAGCAAATCAGTGAATTTTCTAA |
| | | GCGTGTTATTTTAGCAGATGCCAATTTAGATAAAGTTCTTAGTGCATATAACAAACATAGAGAC |
| | | AAACCAATACGTGAACAAGCAGAAAATATTATTCATTTATTTACGTTGACGAATCTTGGAGCTC |
| | | CCGCTGCTTTTAAATATTTTGATACAACAATTGATCGTAAACGATATACGTCTACAAAAGAAGT |
| | | TTTAGATGCCACTCTTATCCATCAATCCATCACTGGTCTTTATGAAACACGCATTGATTTGAGT |
| | | CAGCTAGGAGGTGACGGCAGCGGCAGCAGTATGGTGAGCAAGGGCGAGGAGCTGTTCACC |
| | | GGGGTGGTGCCCATCCTGGTCGAGCTGGACGGCGACGTAAACGGCCACAAGTTCAGCGTGT |
| | | CCGGCGAGGGCGAGGGCGATGCCACCTACGGCAAGCTGACCCTGAAGTTCATCTGCACCAC |
| | | CGGCAAGCTGCCCGTGCCCTGGCCCACCCTCGTGACCACCCTGACCTACGGCGTGCAGTGC |
| | | TTCAGCCGCTACCCCGACCACATGAAGCAGCACGACTTCTTCAAGTCCGCCATGCCCGAAGG |
| | | CTACGTCCAGGAGCGCACCATCTTCTTCAAGGACGACGGCAACTACAAGACCCGCGCCGAG |
| | | GTGAAGTTCGAGGGCGACACCCTGGTGAACCGCATCGAGCTGAAGGGCATCGACTTCAAGG |
| | | AGGACGGCAACATCCTGGGGCACAAGCTGGAGTACAACTACAACAGCCACAACGTCTATATC |
| | | ATGGCCGACAAGCAGAAGAACGGCATCAAGGTGAACTTCAAGATCCGCCACAACATCGAGGA |
| | | CGGCAGCGTGCAGCTCGCCGACCACTACCAGCAGAACACCCCCATCGGCGACGGCCCCGT |
| | | GCTGCTGCCCGACAACCACTACCTGAGCACCCAGTCCGCCCTGAGCAAAGACCCCAACGAG |
| | | AAGCGCGATCACATGGTCCTGCTGGAGTTCGTGACCGCCGCCGGGATCACTCTCGGCATGG |
| | | ACGAGCTGTACAAGTAAAAAAAACAAAAAACAAAACGGCTATTATGCGTTACCGGCGAGACG |
| | | CTACGGACTTAAATAATTGAGCCTTAAAGAAGAAATTCTTTAAGTGGATGCTCTCAAACTCAGG |
| | | GAAACCTAAATCTAGTTATAGACAAGGCAATCCTGAGCCAAGCCGAAGTAGTAATTAGTAAGA |
| | | CCAGTGGACAATCGACGGATAACAGCATATCTAGCGGCCGCGATATC |
| 6 | pUCIDT- | TAATACGACTCACTATAGGGAGACCCTCGACCGTCGATTGTCCACTGGTCAACAATAGATGAC |
| | PIE- | TTACAACTAATCGGAAGGTGCAGAGACTCGACGGGAGCTACCCTAACGTCAAGACGAGGGTA |
| | Aan3.0- | AAGAGAGAGTCCAATTCTCAAAGCCAATAGGCAGTAGCGAAAGCTGCAAGAGAATGAAAATC |
| | T2A-EGFP | CGTTGGCAGCGGCGCCACAAACTTCTCTCTGCTAAAGCAAGCAGGTGATGTTGAAGAAAACC |
| | | CCGGGCCTGGTACTCCCATCATGGTGAGCAAGGGCGAGGAGCTGTTCACCGGGGTGGTGC |
| | | CCATCCTGGTCGAGCTGGACGGCGACGTAAACGGCCACAAGTTCAGCGTGTCCGGCGAGGG |
| | | CGAGGGCGATGCCACCTACGGCAAGCTGACCCTGAAGTTCATCTGCACCACCGGCAAGCTG |
| | | CCCGTGCCCTGGCCCACCCTCGTGACCACCCTGACCTACGGCGTGCAGTGCTTCAGCCGCT |
| | | ACCCCGACCACATGAAGCAGCACGACTTCTTCAAGTCCGCCATGCCCGAAGGCTACGTCCAG |
| | | GAGCGCACCATCTTCTTCAAGGACGACGGCAACTACAAGACCCGCGCCGAGGTGAAGTTCG |
| | | AGGGCGACACCCTGGTGAACCGCATCGAGCTGAAGGGCATCGACTTCAAGGAGGACGGCAA |
| | | CATCCTGGGGCACAAGCTGGAGTACAACTACAACAGCCACAACGTCTATATCATGGCCGACA |
| | | AGCAGAAGAACGGCATCAAGGTGAACTTCAAGATCCGCCACAACATCGAGGACGGCAGCGT |
| | | GCAGCTCGCCGACCACTACCAGCAGAACACCCCCATCGGCGACGGCCCCGTGCTGCTGCCC |
| | | GACAACCACTACCTGAGCACCCAGTCCGCCCTGAGCAAAGACCCCAACGAGAAGCGCGATC |
| | | ACATGGTCCTGCTGGAGTTCGTGACCGCCGCCGGGATCACTCTCGGCATGGACGAGCTGTA |
| | | CAAGACGGACTTAAATAATTGAGCCTTAAAGAAGAAATTCTTTAAGTGGATGCTCTCAAACTCA |
| | | GGGAAACCTAAATCTAGTTATAGACAAGGCAATCCTGAGCCAAGCCGAAGTAGTAATTAGTAA |
| | | GACCAGTGGACAATCGACGGATAACAGCATATCTAGCGGCCGCGATATC |
| 7 | pUCIDT- | TAATACGACTCACTATAGATCGGTCAATCGGTTGGCTTCAGATAACTTACAGCTAATCGGAAG |
| | PIE- | GTGCAGAGACTCGACGGGAGCTACCCTAACGTCAAGACGAGGGTAAAGAGAGAGTCCAATT |
| | Aan3.0- | CTCAAAGCCAATAGGCAGTAGCGAAAGCTGCGGGAGAATGAAAATCCGTTGACCTTAAACGG |
| | 3*Flag | TCGTGTGGGTATTGCTTATATCAGAAATTACACTCGATCTGGACTAAAGCTGCTCATGGATTA |
| | | CAAAGATCACGATGGTGATTATAAAGATCACGACATCGATTACAAGGATGATGATGATAAGGA |
| | | GAAGATTCATCCTCCTTCAATGCCTGGAGATTTCTGAGCTAAGCAAGCAAGACGCTACGGACT |
| | | TAAATAATTGAGCCTTAGAGAAGAAATTCTTTAAGTGGATGCTCTCAAACTCAGGGAAACCTAA |
| | | ATCTAGCTATAGACAAGGCAATCCTGAGCCAAGCCGAAGTAGTAATTAGTAAGTTAACAAGCG |
| | | CCAACCGATTGACCGATATCAG |
| 8 | Primer: | AGACGCTACGGACTTAAATAATTGAGC |
| | empty-vF | |
| 9 | Primer: | GTTTAAGGTCAACGGATTTTCATTCTC |
| | empty-vR | |
| 10 | Primer: | AAAATCCGTTGACCTTAAACGGTCG |
| | CVB3- | |
| | EGFP-gF | |
| | (F1) | |
| 11 | Primer: | AAGTCCGTAGCGTCTCGCC |
| | CVB3- | |
| | EGFP-gR | |
| 12 | Primer: | GTTGAATACAGCAAAATGGAAGATGCCAAAAACATTAAGAAGG |
| | CVB3- | |
| | Fluci-gF | |
| 13 | Primer: | TTTGTTTTTTTTACTTATCATCATCATCCTTGTAATCACTGCTCACGGCGATCTTGCCGC |
| | CVB3- | |
| | Fluci-gR | |
| 14 | Primer: | AGTAAAAAAAACAAAAAACAAAACGGCTATTATGCGTTACCGGCGAGACGCTACGGACTT |
| | CVB3- | |
| | Fluci-vF | |
| | (F2) | |
| 15 | Primer: | TTTGCTGTATTCAACTTAACAATGAATTGTAATG |
| | CVB3- | |
| | Fluci/Spike/ | |
| | Cas9-vR | |
| | (R) | |
| 16 | Primer: | GTTGAATACAGCAAAATGGGGATTCTGCCTAGCCC |
| | CVB3- | |
| | Spike- | |
| | EGFP-gF | |
| 17 | Primer: | ACTGCTGCCGCTGCCCAAGAAGGTGCTCAGCAGCAC |
| | CVB3- | |
| | Spike- | |
| | EGFP-gR | |
| 18 | Primer: | GTTGAATACAGCAAAATGGATAAGAAATACTCAATAGGCTTAGATATC |
| | CVB3- | |
| | Cas9- | |
| | EGFP-gF | |
| 19 | Primer: | ACTGCTGCCGCTGCCGTCACCTCCTAGCTGACTCAAATC |
| | CVB3- | |
| | Cas9- | |
| | EGFP-gR | |
| 20 | Primer: | GGCAGCGGCAGCAGTATGGTGAGCAAGGGCGAGG |
| | Spike/Cas9- | |
| | vF | |
| 21 | Primer: | CTGCTAAAGCAAGCAGGTGATGTTGAAGAAAACCCCGGGCCTGGTACTCCCATCATGGTGAG |
| | T2A-EGFP- | CAAGGGCGAG |
| | gF | |
| 22 | Primer: | ATTATTTAAGTCCGTCTTGTACAGCTCGTCCATGC |
| | T2A-EGFP- | |
| | gR | |
| 23 | Primer: | ACGGACTTAAATAATTGAGCC |
| | T2A-EGFP- | |
| | vF | |
| 24 | Primer: | TGCTTGCTTTAGCAGAGAGAAGTTTGTGGCGCCGCTGCCAACGGATTTTCATTCTCTTGC |
| | T2A-EGFP- | |
| | vR | |

### REFERENCES

Zaug A J et al, McEvoy M M and Cech T R. Self-splicing of the Group I Intron from Anabaena Pre-tRNA Requirement for Base-Pairing of the Exons in the Anticodon Stem. Biochemistry vol. 32 (31) p 7946-53 (1993)
Wesselhoeft, R. A., Kowalski, P. S. and Anderson, D. G. Engineering circular RNA for potent and stable translation in eukaryotic cells. Nat Commun vol. 9, p 2629 (2018)
Abe BT, Wesselhoeft RA, Chen R, Anderson DG and Chang HY. Circular RNA migration in agarose gel electrophoresis. Molecular Cell vol. 82 p 1768-1777 (2022)
Zhang Y, Yang L and Chen LL. Characterization of Circular RNAs. In Feny Y and Zhang L (eds) Long Non-Coding RNA: Methods and Protocol, Methods in Molecular Biology, vol 1402 (2016)
Masek, T., Vopalensky, V., Suchomelova, P. & Pospisek, M. Denaturing RNA electrophoresis in TAE agarose gels. Anal Biochem 336, (2005)
Sumitomo, K., Sasaki, M. & Yamaguchi, Y. Acetic acid denaturing for RNA capillary polymer electrophoresis. Electrophoresis 30, 1538-1543, (2009).
Rosen, J. M., Woo, S. L., Holder, J. W., Means, A. R. & O'Malley, B. W. Preparation and preliminary characterization of purified ovalbumin messenger RNA from the hen oviduct. Biochemistry-Us 14, 69-78 (1975).
Reijnders, L., Sloof, P., Sival, J. & Borst, P. Gel electrophoresis of RNA under denaturing conditions. Biochim Biophys Acta 324, 320-333 (1973)
Rio, D. C. Denaturation and electrophoresis of RNA with formaldehyde. Cold Spring Harbor Protocols, p 219-222, (2015)

1. A method of separating RNA present in a sample, the method comprising:
(a) loading an electrophoresis gel with the RNA-containing sample, wherein the electrophoresis gel is an urea-agarose gel; and
(b) applying an electric field across the gel to cause the RNA to migrate across the gel, wherein linear and circular RNA present in the sample migrate through the gel at different speeds.

2. The method according to claim 1 wherein the gel comprises urea at a concentration of about 1M to about 8M.

3. The method according to claim 1 or 2 wherein the gel comprises urea at a concentration of about 4M to 6M.

4. The method according to any one of claims 1 to 3 wherein the agarose is present at a concentration of about 0.5% to 6% agarose.

5. The method according to any one of claims 1 to 4 wherein the agarose is present at a concentration of about 1.5% agarose.

6. The method according to any one of claims 1 to 5 wherein the electric field is run at about 15W-40W.

7. The method according to any one of claims 1 to 6 wherein the electric field is run at about 15W-25W.

8. The method according to any one of claims 1 to 7 wherein the electric field is run for a period of about 10-40 minutes.

9. The method according to any one of claims 1 to 8 wherein the electric field is run for a period of about 10-30 minutes.

10. The method according to any one of claims 1 to 9 wherein the gel is a 6M urea/1.5% agarose gel.

11. The method according to any one of claims 1 to 10 wherein the electric field is run at about 15-25W for a time period of about 10-30 minutes.

12. The method according to any one of claims 1 to 11 wherein the method is for separating RNA having a length up to about 6000 nucleotides.

13. The method according to any one of claims 1 to 12 wherein the method is for resolving RNA having a length in the range of about 200 to about 6000 nucleotides.

14. The method according to any one of claims 1 to 9 wherein the method is for resolving RNA having a length in the range of about 500 to about 6000 nucleotides, wherein the gel is a 4M urea/1.5% agarose gel, and the method comprises running the electric field at a constant power of about 20W for 10-30 minutes.

15. The method according to any one of claims 1 to 14 wherein a vertical electrophoresis gel system is used.

16. The method according to any one of claims 1 to 15 wherein the method further comprises:
(a) running in a first lane of the electrophoresis gel a control sample comprising a molecular weight marker;
(b) running in a second lane of the electrophoresis gel the RNA sample; and
(c) visualising the bands.

17. The method according to any one of claims 1 to 16 wherein bands of the gel are visualised by UV imaging techniques.

18. The method according to any one of claims 1 to 17, wherein the method further comprises the step of mixing the RNA sample with a loading buffer, before loading the gel with the sample, wherein the loading buffer comprises a denaturing agent.

19. The method according to claim 18 wherein the denaturing agent of the loading buffer is urea.

20. A method for separating circular RNA from linear RNA in a sample, the method comprising electrophoretically separating RNA in sample in a gel, wherein the gel is a denaturing urea-agarose gel and positioned in a vertical arrangement.

21. The method according to claim 20 wherein:
(a) the urea-agarose gel as defined in any one of claims 1 to 19; and/or
(b) electrophoretically separating RNA in sample in a gel, comprises running the gel as defined in any one of claims 1 to 19.

22. A method for analysing RNA present in a sample, the method comprising analysing the sample by gel electrophoresis, wherein the wherein the gel is a denaturing urea-agarose gel and wherein the method comprises using a vertical gel electrophoresis system.

23. The method according to claim 22 wherein:
(a) the urea-agarose gel as defined in any one of claims 1 to 19; and/or
(b) analysing the sample by gel electrophoresis, comprises running the gel as defined in any one of claims 1 to 19.

24. A kit for separating linear and circular RNA in a sample, the kit comprising
(a) components for carrying out gel electrophoresis of a RNA containing sample, wherein the components include agarose and urea for the preparation of a urea-agarose gel as defined in any one of claims 1 to 19;
(b) a loading buffer for mixing with the RNA sample, wherein the loading buffer comprises urea as a denaturing agent; and
(c) a standard molecular weight control sample.

25. The kit according to claim 24 wherein the kit further comprises an apparatus for vertical gel electrophoresis.
